(19) United States
(12) Patent Application Publication
(10) Pub. No.: US 2024/0024048 A1
(43) Pub. Date: Jan. 25, 2024

(54) SYSTEMS AND METHODS FOR ESTABLISHING PROCEDURAL SETUP OF ROBOTIC MEDICAL SYSTEMS

(71) Applicant: Auris Health, Inc., Redwood City, CA (US)

(72) Inventors: Yanan HUANG, Sunnyvale, CA (US); Thai Chau NGUYEN HUYNH, Sunnyvale, CA (US); Vahid NAZARI, San Jose, CA (US); Caitlin Marie ROMANCZYK, San Francisco, CA (US); Alexander Tarek HASSAN, San Francisco, CA (US)

(21) Appl. No.: 18/474,005

(22) Filed: Sep. 25, 2023

Related U.S. Application Data
(63) Continuation of application No. PCT/IB2022/051733, filed on Feb. 28, 2022.
(60) Provisional application No. 63/166,969, filed on Mar. 26, 2021.

(51) Int. Cl. A61B 34/32; A61B 34/00; A61B 34/35; G05B 19/4155; B25J 9/16

(52) U.S. Cl. CPC A61B 34/32; A61B 34/25; A61B 34/35; G05B 19/4155; B25J 9/1664; A61B 2034/2059

(57) ABSTRACT

Robotic medical systems can be capable of establishing procedural setup. A robotic medical system can include a kinematic chain having at least a first robotic arm. The robotic medical system can be configured to execute first movement of the kinematic chain to a first pose in accordance with a first recommended pose corresponding to a first procedure to be performed on a patient. After the kinematic chain reaches the first pose, the robotic medical system can obtain first data corresponding to a boundary condition of the kinematic chain in accordance with an input from a user and/or second data corresponding to a current state of the patient. The robotic medical system can be configured to adjust at least a portion of the kinematic chain from the first pose to a second pose in accordance with the obtained first data and/or second data.

### CROSS-REFERENCE TO RELATED APPLICATIONS

This present application is a continuation of International Patent Application PCT/IB2022/051733 filed Feb. 28, 2022 and entitled “SYSTEMS AND METHODS FOR ESTABLISHING PROCEDURAL SETUP OF ROBOTIC MEDICAL SYSTEMS,” which claims priority to U.S. Provisional Application No. 63/166,969 filed Mar. 26, 2021 and entitled, “SYSTEMS AND METHODS FOR ESTABLISHING PROCEDURAL SETUP OF ROBOTIC MEDICAL SYSTEMS,” both of which are incorporated herein by reference in their entirety for all purposes.

### TECHNICAL FIELD

The systems and methods disclosed herein are directed to robotic medical systems, and more particularly to configuring robotically controlled arms of robotic medical systems for medical procedures.

### BACKGROUND

A robotically-enabled medical system is capable of performing a variety of medical procedures, including both minimally invasive, such as laparoscopy, and non-invasive, such as endoscopy, procedures. Among endoscopic procedures, the system may be capable of performing bronchoscopy, ureteroscopy, gastroscopy, etc.

Such robotic medical systems may include robotic arms configured to control the movement of medical tool(s) during a given medical procedure. In order to achieve a desired pose of a medical tool, a robotic arm may be placed into a pose during a set-up process or during teleoperation. Some robotically-enabled medical systems may include an arm support (e.g., a bar) that is connected to respective bases of the robotic arms and supports the robotic arms.

### SUMMARY

One of the biggest challenges for robotic medical systems is to set up a procedure properly so that the robotic surgery can be executed smoothly with minimal interruptions. A robotic surgery can be interrupted due to many factors, including workspace boundaries, arm collisions, kinematic singularities, internal instrument conflicts, triangulation limitations, etc. The level of difficulty of setting up a procedure also varies depending the type of the procedure, the applied surgical techniques, and patient conditions such as patient size, fixation, etc.

Accordingly, an improved robotic medical system is desirable. In particular, there is a need for a robotic medical system that can provide the design and delivery of an accurate procedural setup that reduce unnecessary and/or dangerous disruptions during surgery.

As disclosed herein, a robotic medical system is configured to refine and optimize a pose (e.g., position and/or orientation) of adjustable arm support(s) (also referred to as “bar(s)”) and/or robotic arm(s) of the system during set-up by taking into consideration a recommended bar and/or arm pose based on a procedure selection and at least one of a boundary condition established by the user (e.g., accessory consideration) or patient information inferred from one or more port locations, in accordance with some embodiments.

In another aspect of the present disclosure, a robotic system is configured to gather additional information (e.g., based on the port locations) after the robotic arms are placed in a docked state. The robotic system further optimizes the pose of the robotic arms and/or one or more underlying bars based on the additional information.

In another aspect of the present disclosure, a robotic system includes a user interface that enables a user to supervise adjustments from an actual port location to a recommended port location, or from actual bar and/or arm poses to system-recommended bar and/or arm poses. In some embodiments, the robotic system also provides comparisons (e.g., visual comparisons, visual representations of the results of the comparisons, etc.) between the actual and recommended port locations and/or between the actual and recommended bar and/or arm poses to assist user decisions regarding the adjustments of the port location.

Accordingly, the systems and/or methods disclosed herein advantageously improves patient and/or operator safety during surgery. It also reduces interruptions while the surgeon is driving one or more of the robotic arms (e.g., via teleoperation) during surgery.

The systems, methods and devices of this disclosure each have several innovative aspects, no single one of which is solely responsible for the desirable attributes disclosed herein.

In accordance with some embodiments of the present disclosure, a robotic system includes a kinematic chain that includes at least a first robotic arm. The robotic system also includes one or more processors and memory. The memory stores instructions that, when executed by the one or more processors, cause the one or more processors to execute first movement of the kinematic chain to a first pose in accordance with a first recommended pose. The first recommended pose corresponds to a first procedure to be performed on a patient. The memory also stores instructions that, when executed by the one or more processors, cause the one or more processors to: after the kinematic chain reaches the first pose by executing the first movement, obtain first data corresponding to a boundary condition of the kinematic chain in accordance with an input from a user and/or second data corresponding to a current state of the patient. The memory also stores instructions that, when executed by the one or more processors, cause the one or more processors to adjust at least a portion of the kinematic chain from the first pose to a second pose in accordance with the obtained first data and/or second data.

In some embodiments, the memory further stores one or more recommended poses for the kinematic chain, corresponding to one or more procedural setups. The one or more recommended poses include the first pose.

In some embodiments, the memory further includes instructions that, when executed by the one or more processors, cause the one or more processors to: prior to storing the one or more recommended poses, generate the one or more recommended poses.

In some embodiments, the memory further includes instructions that, when executed by the one or more processors, cause the one or more processors to: after the kinematic chain reaches the first pose by executing the first movement, place the first robotic arm in a docked state.

In some embodiments, the memory further includes instructions that, when executed by the one or more processors, cause the one or more processors to: in accordance with a determination that the first robotic arm is in the docked state, determine a port of entry and/or remote center of motion (RCM) with respect to the first robotic arm.

In some embodiments, the boundary condition of the kinematic chain is established by a user.

In some embodiments, the boundary condition of the kinematic chain is determined based on a respective position of one or more accessories in a vicinity of the kinematic chain.

In some embodiments, the boundary condition of the kinematic chain is determined based on a respective position of one or more accessories attached to a support platform of the robotic system.

In some embodiments the first data corresponding to the boundary condition of the kinematic chain is based on positioning and/or fixation of the patient on the robotic system.

In some embodiments the positioning and/or fixation of the patient is inferred from one or more port locations corresponding to the kinematic chain.

In accordance with some embodiments of the present disclosure, a robotic system includes a display. The robotic system also includes a kinematic chain. The robotic system also includes one or more processors and memory. The memory stores instructions that, when executed by the one or more processors, cause the one or more processors to: after the kinematic chain has entered a docked state, determine an actual pose of the kinematic chain and/or an actual port location corresponding to the kinematic chain. The memory stores instructions that, when executed by the one or more processors, cause the one or more processors to generate a recommended pose for the kinematic chain and/or a first recommended port location corresponding to the kinematic chain in accordance with a first procedural selection. The memory stores instructions that, when executed by the one or more processors, cause the one or more processors to compare the recommended pose with the actual pose and/or the first recommended port location with the actual port location. The memory stores instructions that, when executed by the one or more processors, cause the one or more processors to: in accordance with the comparison, determine that a difference between the recommended pose and the actual pose and/or a difference between the first recommended port location and the actual port location meet first criteria, the first criteria including a first threshold amount of difference. The memory also stores instructions that, when executed by the one or more processors, cause the one or more processors to generate a first notification regarding the difference between the recommended pose and the actual pose and/or the difference between the first recommended port location and the actual port location, and output the first notification.

In some embodiments, the kinematic chain includes a first robotic arm that is in the docked state.

In some embodiments, generating the first notification includes generating a first visualization that includes (1) the actual pose and the recommended pose and/or (2) the actual port location and the first recommended port location. The memory further includes instructions that, when executed by the one or more processors, cause the one or more processors to display the first visualization on a user interface of the robotic system.

In some embodiments, comparing the first recommended port location with the actual port location includes: determining a separation distance between the first recommended port location and the actual port location, and comparing the separation distance with one or more preset margins.

In some embodiments, the one or more preset margins include a first preset margin and a second preset margin.

In some embodiments, the first preset margin is between 3 and 10 millimeters.

In some embodiments, the second preset margin is between 0 and 6 millimeters.

In some embodiments, the memory further includes instructions that, when executed by the one or more processors, cause the one or more processors to: in accordance with a determination that the separation distance exceeds the first preset margin, display on a user interface a first recommendation to manually adjust a port location from the actual location to a first location that is within the first preset margin.

In some embodiments, the memory further includes instructions that, when executed by the one or more processors, cause the one or more processors to: in accordance with a determination that the separation distance is within the first preset margin, determine whether movement of the kinematic chain from the actual pose to the recommended pose is within a pre-established movement boundary. The memory includes instructions that, when executed by the one or more processors, cause the one or more processors to: in accordance with a determination that the movement of the kinematic chain from the actual pose to the recommended pose exceeds the pre-established movement boundary, display on a user interface a second recommendation for a user to manually adjust the kinematic chain from the actual pose to a second pose. The memory includes instructions that, when executed by the one or more processors, cause the one or more processors to: in accordance with a determination that the movement of the kinematic chain from the actual pose to the recommended pose is within the pre-established movement boundary, determine whether the separation distance is within the second preset margin.

In some embodiments, the memory further includes instructions that, when executed by the one or more processors, cause the one or more processors to: in accordance with a determination that the separation distance is within the second preset margin, generate a second visualization that includes the actual port location and the first recommended port location. The memory also includes instructions that, when executed by the one or more processors, cause the one or more processors to generate a second notification requesting user confirmation that it is safe to move a port location from the actual port location to the first recommended port location, and display the second visualization, the second notification, and a first interface element corresponding to the second notification on the user interface.

In some embodiments, the memory further includes instructions that, when executed by the one or more processors, cause the one or more processors to receive user selection of the first interface element. The memory also includes instructions that, when executed by the one or more processors, cause the processors to: in response to the user selection, generate and display, on the user interface, a second interface element. User selection of the second interface element causes the robotic system to automatically execute a first movement to move the port location from the actual port location to the first recommended port location.

In some embodiments, the kinematic chain comprises a first robotic arm that is in the docked state. The memory further includes instructions that, when executed by the one or more processors, cause the one or more processors to: in accordance with executing the first movement to move the port location from the actual port location to the first recommended port location, adjust a remote center of motion (RCM) with respect to the first robotic arm based on the first recommended port location.

In some embodiments, adjusting the remote center of motion (RCM) comprises moving at least a portion of the first robotic arm and/or an adjustable bar of the kinematic chain relative to a support platform of the robotic system.

In some embodiments, the memory further includes instructions that, when executed by the one or more processors, cause the one or more processors to: in accordance with a determination that the separation distance exceeds the second margin, generate and display on the user interface a third visualization that includes (1) the actual pose and the first recommended pose and/or (2) the actual port location and the first recommended port location. The memory includes instructions that, when executed by the one or more processors, cause the one or more processors to generate and display on the user interface a third interface element. User selection of the third interface element causes the robotic system to automatically execute a first movement to move a port location from the actual port location to the first recommended port location.

In some embodiments, generating the recommended pose comprises determining whether the recommended pose meets a boundary condition.

In some embodiments, the boundary condition is based on a respective location of one or more accessories and/or patient fixation.

In some embodiments, the memory further includes instructions that, when executed by the one or more processors, cause the one or more processors to: in accordance with a determination that the recommended pose does not meet the boundary condition, generate and display on the user interface a third notification that includes information of the difference between the actual pose and the first recommended pose exceeds the boundary condition. The memory also includes instructions that, when executed by the one or more processors, cause the one or more processors to generate and display a request to adjust the kinematic chain.

In some embodiments, the memory further includes instructions that, when executed by the one or more processors, cause the one or more processors to: in accordance with a determination that the recommended pose exceeds the boundary condition, generate and execute a modified target pose that meets the boundary condition.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy procedure(s).

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopy procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopy procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance to an example embodiment.

FIG. 21 illustrates an exemplary robotic system according to some embodiments.

FIG. 22 illustrates another view of an exemplary robotic system according to some embodiments.

FIGS. 23A to 23C illustrate different views of an exemplary robotic arm according to some embodiments.

FIG. 24 illustrates a setup workflow for a robotic system in accordance with some embodiments.

FIG. 25 illustrates a robotic system in a stowed state in accordance with some embodiments.

FIGS. 26A and 26B illustrate respectively, a robotic system in an initial deployed state and arms and bar of the robotic system covered by drapes, in accordance with some embodiments.

FIG. 27 illustrates a robotic system that includes one set of robotic arms in a pre-docking pose and another set of robotic arms that are being robotically moved to a pre-docking pose, in accordance with some embodiments.

FIG. 28 illustrates an exemplary setup for a surgery that is performed using a robotic system, in accordance with some embodiments.

FIG. 29 illustrates a flow diagram for determining a desired bar and/or arm pose through continuous activation in accordance with some embodiments.

FIG. 30 illustrates an exemplary user interface depicting comparisons between actual and recommended bar and/or arm poses, in accordance with some embodiments.

FIGS. 31A and 31B illustrate a flowchart diagram for a method 1100 for procedural setup performed by one or more processors of a robotic system, in accordance with some embodiments.

FIGS. 32A to 32D illustrate a flowchart diagram for a method 1200 performed by one or more processors of a robotic, in accordance with some embodiments.

### DETAILED DESCRIPTION

### 1. Overview.

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other embodiments of the disclosed concepts are possible, and various advantages can be achieved with the disclosed embodiments. Headings are included herein for reference and to aid in locating various sections. These headings are not intended to limit the scope of the concepts described with respect thereto. Such concepts may have applicability throughout the entire specification.

### A. Robotic System—Cart.

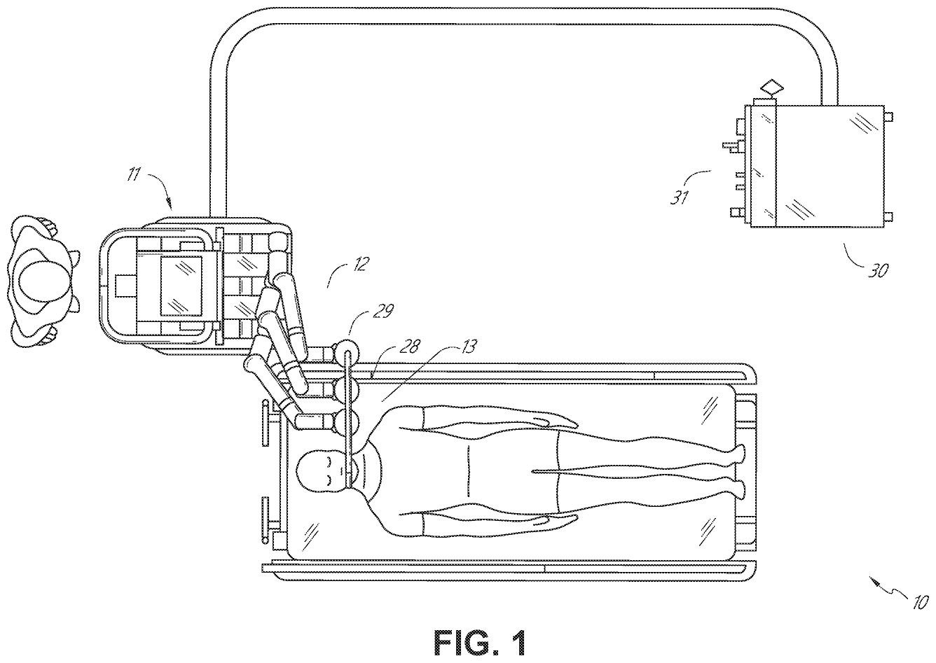

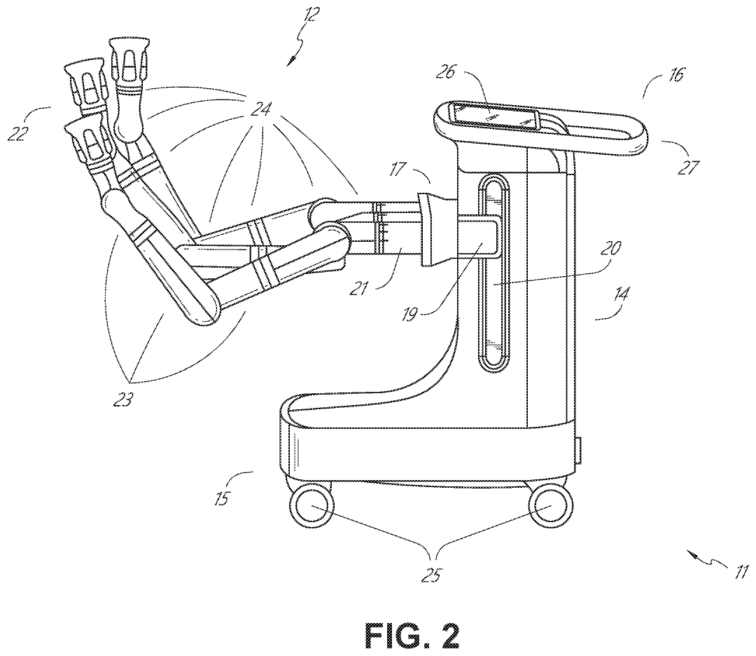

The robotically-enabled medical system may be configured in a variety of ways depending on the particular procedure. FIG. 1 illustrates an embodiment of a cart-based robotically-enabled system 10 arranged for a diagnostic and/or therapeutic bronchoscopy procedure. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independent of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include opto-electronics equipment for detecting, receiving, and processing data received from the optical sensors or cameras throughout the robotic system 10. In combination with the control system, such opto-electronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in system 10 are generally designed to provide both robotic controls as well as pre-operative and real-time information of the procedure, such as navigational and localization information of the endoscope 13. When the console 31 is not the only console available to the physician, it may be used by a second operator, such as a nurse, to monitor the health or vitals of the patient and the operation of system, as well as provide procedure-specific data, such as navigational and localization information. In other embodiments, the console 30 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart, the support for controls, optics, fluidics, and/or navigation may be provided through a separate cable.

FIG. 2 provides a detailed illustration of an embodiment of the cart from the cart-based robotically-enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm. Each of the arms 12 have seven joints, and thus provide seven degrees of freedom. A multitude of joints result in a multitude of degrees of freedom, allowing for “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and arms 12 over the floor. Accordingly, the cart base 15 houses heavier components, such as electronics, motors, power supply, as well as components that either enable movement and/or immobilize the cart. For example, the cart base 15 includes rollable wheel-shaped casters 25 that allow for the cart to easily move around the room prior to a procedure. After reaching the appropriate position, the casters 25 may be immobilized using wheel locks to hold the cart 11 in place during the procedure.

Positioned at the vertical end of column 14, the console 16 allows for both a user interface for receiving user input and a display screen (or a dual-purpose device such as, for example, a touchscreen 26) to provide the physician user with both pre-operative and intra-operative data. Potential pre-operative data on the touchscreen 26 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 16 may be positioned and tilted to allow a physician to access the console from the side of the column 14 opposite carriage 17. From this position, the physician may view the console 16, robotic arms 12, and patient while operating the console 16 from behind the cart 11. As shown, the console 16 also includes a handle 27 to assist with maneuvering and stabilizing cart 11.

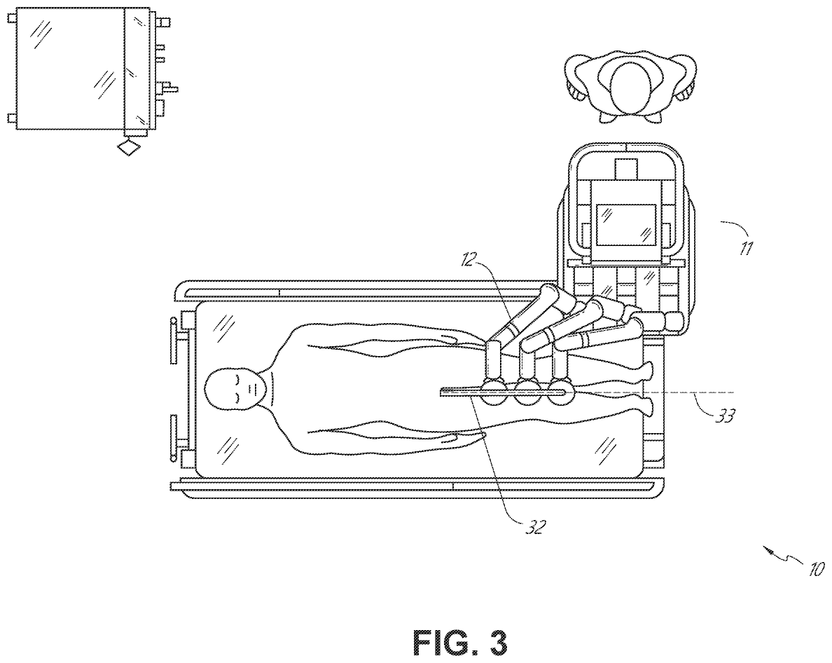

FIG. 3 illustrates an embodiment of a robotically-enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

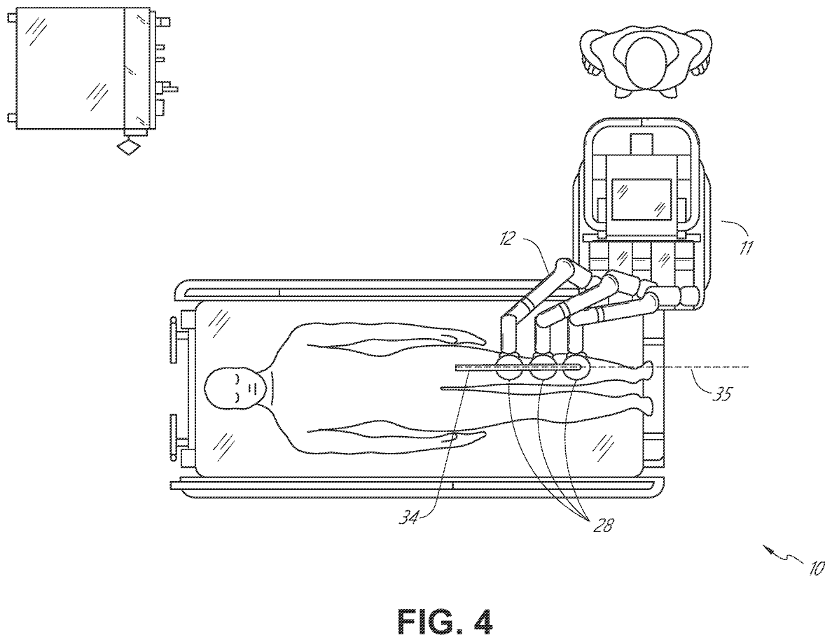

FIG. 4 illustrates an embodiment of a robotically-enabled system similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table.

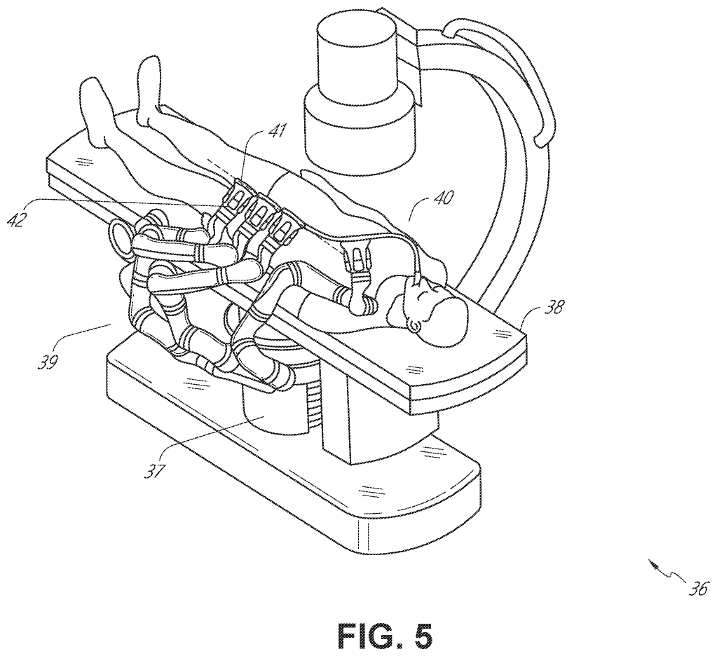

Embodiments of the robotically-enabled medical system may also incorporate the patient's table. Incorporation of the table reduces the amount of capital equipment within the operating room by removing the cart, which allows greater access to the patient. FIG. 5 illustrates an embodiment of such a robotically-enabled system arranged for a bronchoscopy procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around table 38.

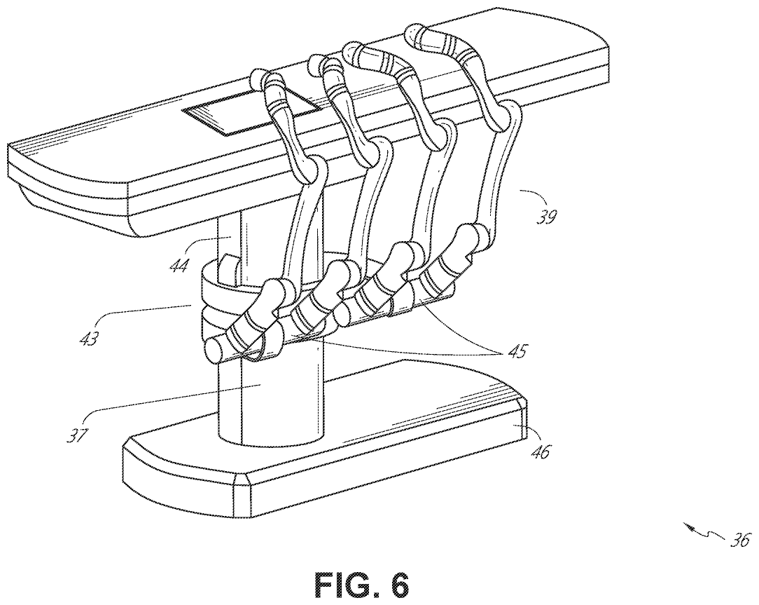

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independent of the other carriages. While carriages 43 need not surround the column 37 or even be circular, the ring-shape as shown facilitates rotation of the carriages 43 around the column 37 while maintaining structural balance. Rotation and translation of the carriages 43 allows the system to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

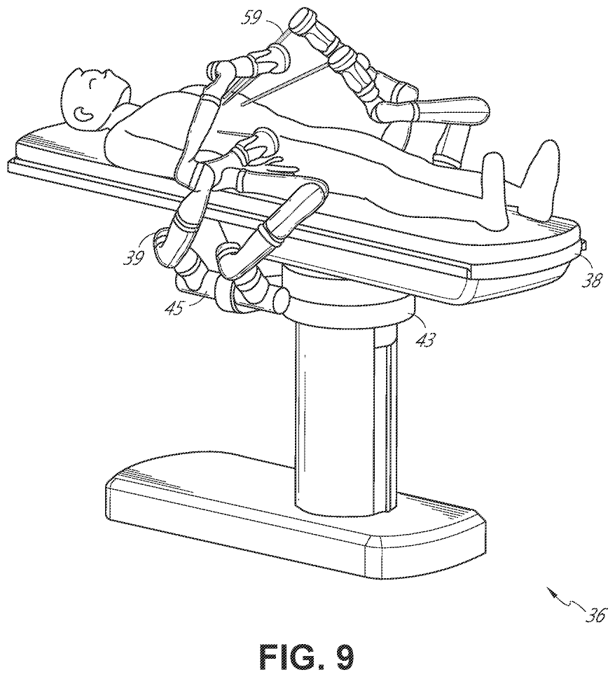

The arms 39 may be mounted on the carriages through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of table 38 (as shown in FIG. 6), on opposite sides of table 38 (as shown in FIG. 9), or on adjacent sides of the table 38 (not shown).

The column 37 structurally provides support for the table 38, and a path for vertical translation of the carriages. Internally, the column 37 may be equipped with lead screws for guiding vertical translation of the carriages, and motors to mechanize the translation of said carriages based the lead screws. The column 37 may also convey power and control signals to the carriage 43 and robotic arms 39 mounted thereon.

The table base 46 serves a similar function as the cart base 15 in cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

Continuing with FIG. 6, the system 36 may also include a tower (not shown) that divides the functionality of system 36 between table and tower to reduce the form factor and bulk of the table. As in earlier disclosed embodiments, the tower may provide a variety of support functionalities to table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base for potential stowage of the robotic arms. The tower may also include a master controller or console that provides both a user interface for user input, such as keyboard and/or pendant, as well as a display screen (or touchscreen) for pre-operative and intra-operative information, such as real-time imaging, navigation, and tracking information. In some embodiments, the tower may also contain holders for gas tanks to be used for insufflation.

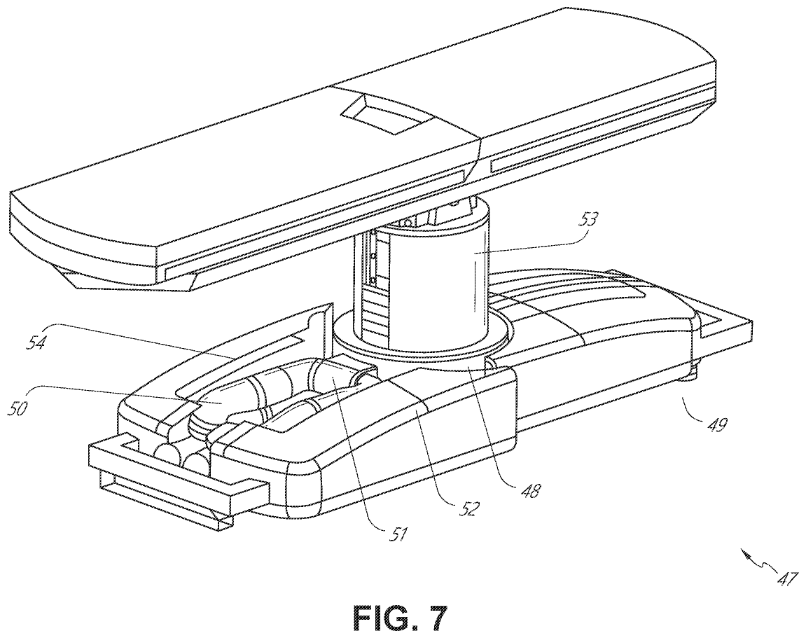

In some embodiments, a table base may stow and store the robotic arms when not in use. FIG. 7 illustrates a system 47 that stows robotic arms in an embodiment of the table-based system. In system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and arms 50 around column 53, and closed to stow to protect them when not in use. The base covers 52 may be sealed with a membrane 54 along the edges of its opening to prevent dirt and fluid ingress when closed.

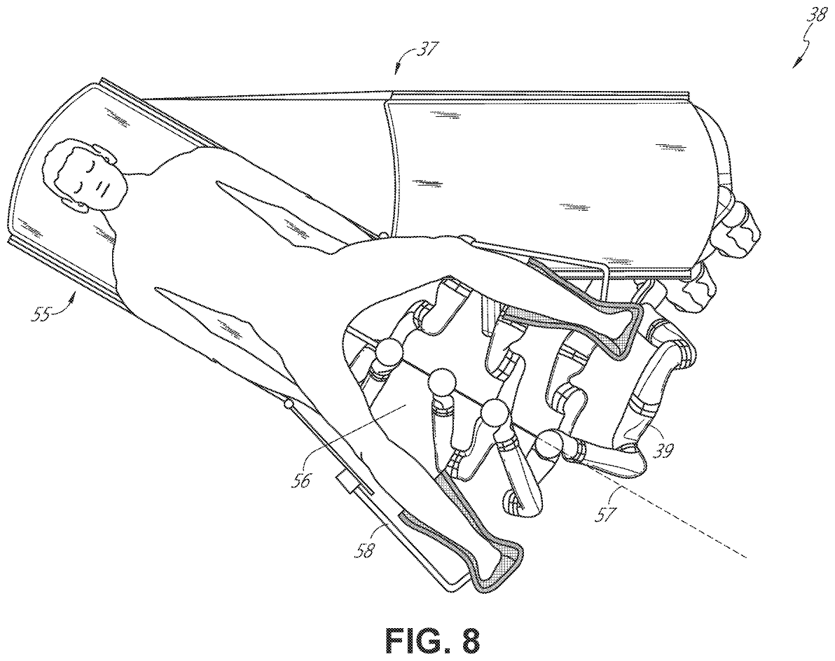

FIG. 8 illustrates an embodiment of a robotically-enabled table-based system configured for a ureteroscopy procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage 35 (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically-enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

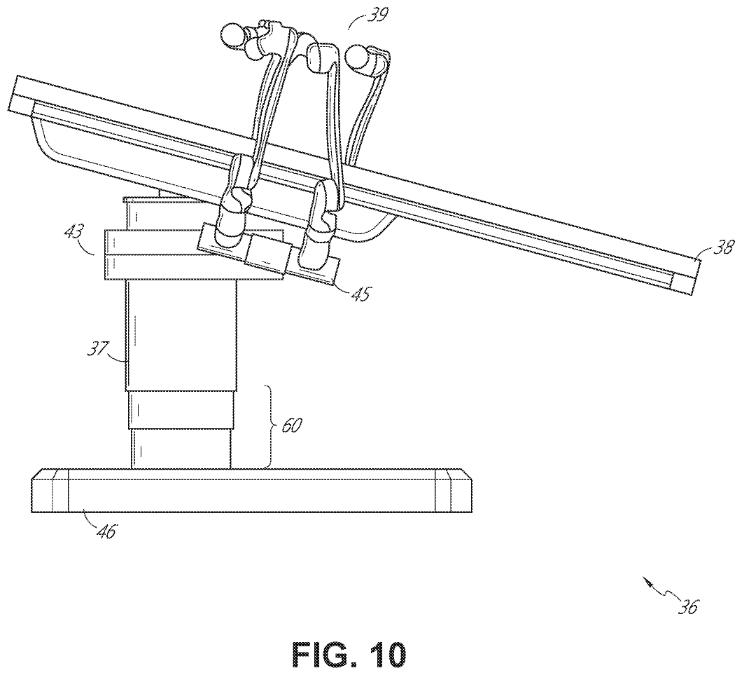

To accommodate laparoscopic procedures, the robotically-enabled table system may also tilt the platform to a desired angle. FIG. 10 illustrates an embodiment of the robotically-enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the arms 39 maintain the same planar relationship with table 38. To accommodate steeper angles, the column 37 may also include telescoping portions 60 that allow vertical extension of column 37 to keep the table 38 from touching the floor or colliding with base 46.

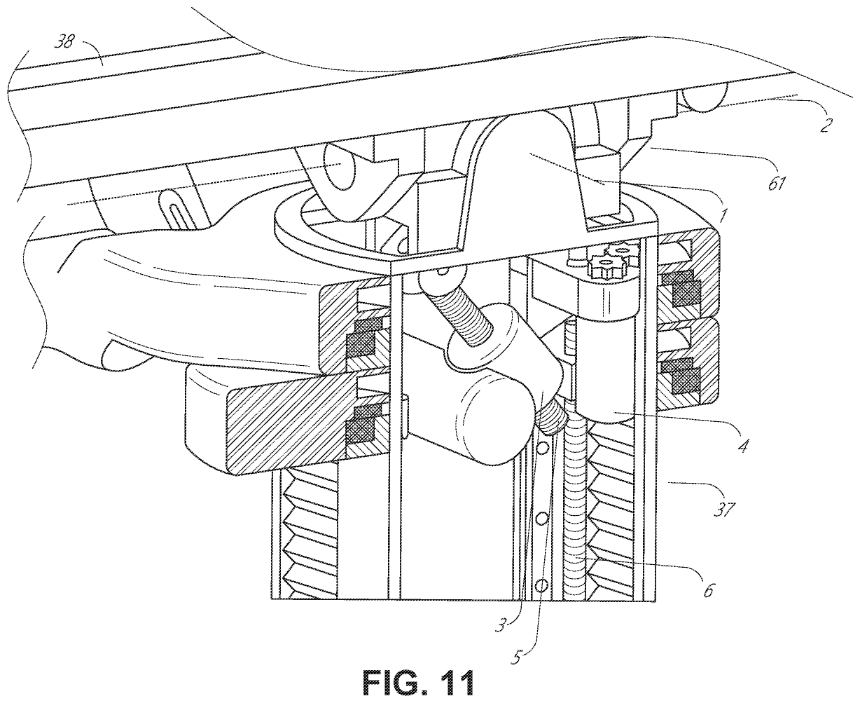

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's lower abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

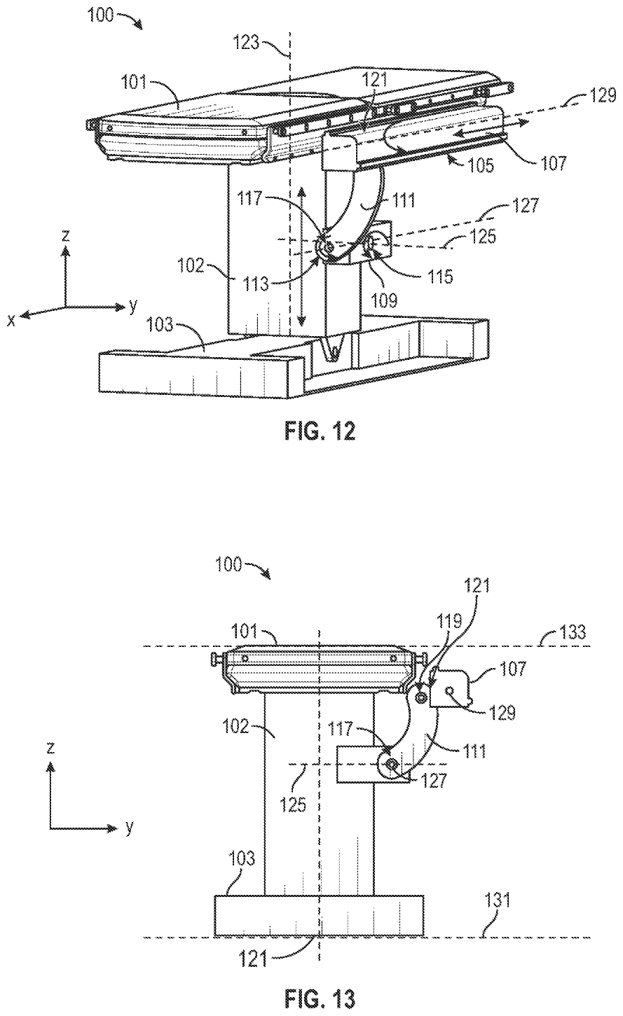

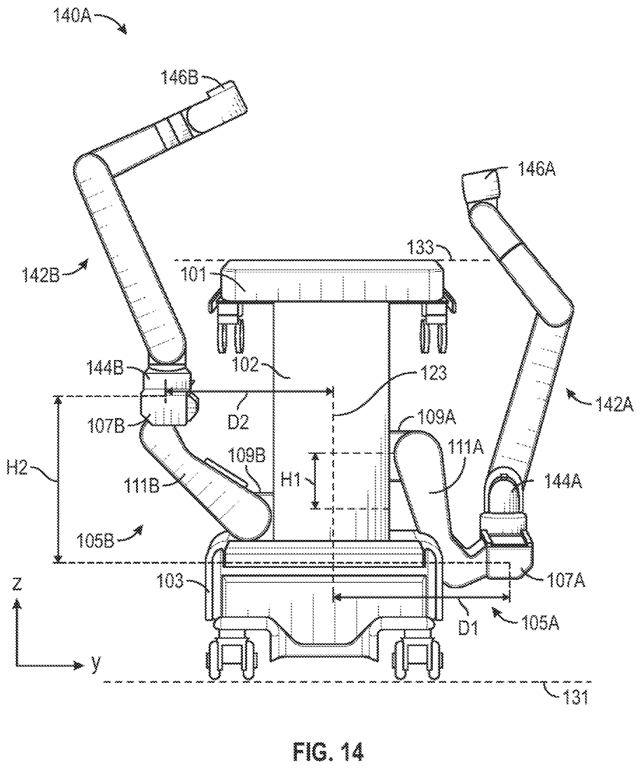

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (“Z-lift”) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface.

The end effectors of the system's robotic arms comprise (i) an instrument driver (alternatively referred to as “instrument drive mechanism” or “instrument device manipulator”) that incorporate electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

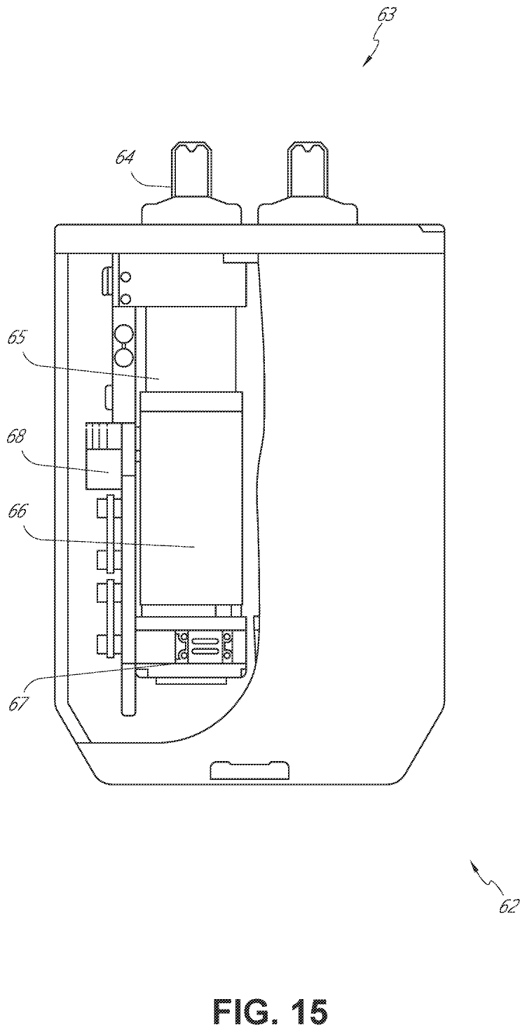

FIG. 15 illustrates an example instrument driver. Positioned at the distal end of a robotic arm, instrument driver 62 comprises of one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuitry 68 for receiving control signals and actuating the drive unit. Each drive unit 63 being independent controlled and motorized, the instrument driver 62 may provide multiple (four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise of a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

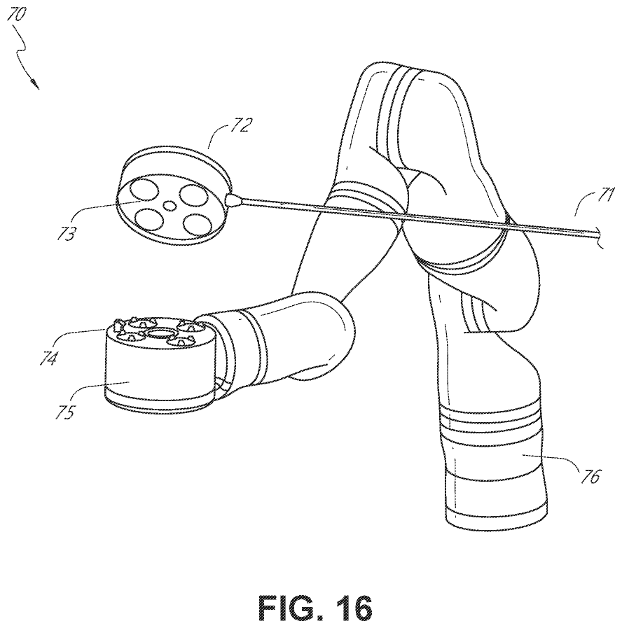

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of instrument base 72 may share axes of rotation with the drive outputs 74 in the instrument driver 75 to allow the transfer of torque from drive outputs 74 to drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at distal end of the elongated shaft 71, where tension from the tendon cause the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft may comprise of a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include of an optical camera. The shaft 71 may also accommodate optical fibers to carry light from proximally-located light sources, such as light emitting diodes, to the distal end of the shaft.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft during an endoscopic procedure.

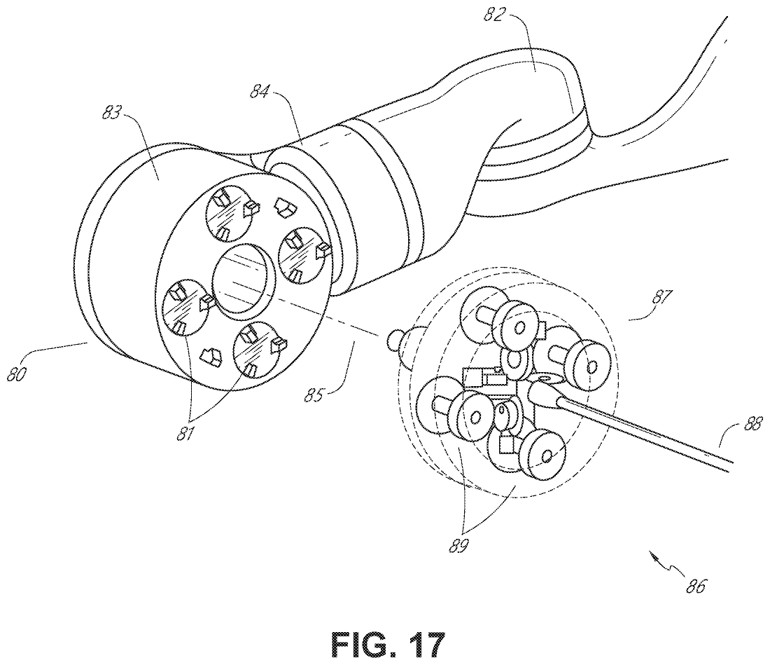

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver. Power and controls signals may be communicated from the non-rotational portion 84 of the instrument driver 80 to the rotational assembly 83 through electrical contacts and may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, instrument shaft 88 extends from the center of instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

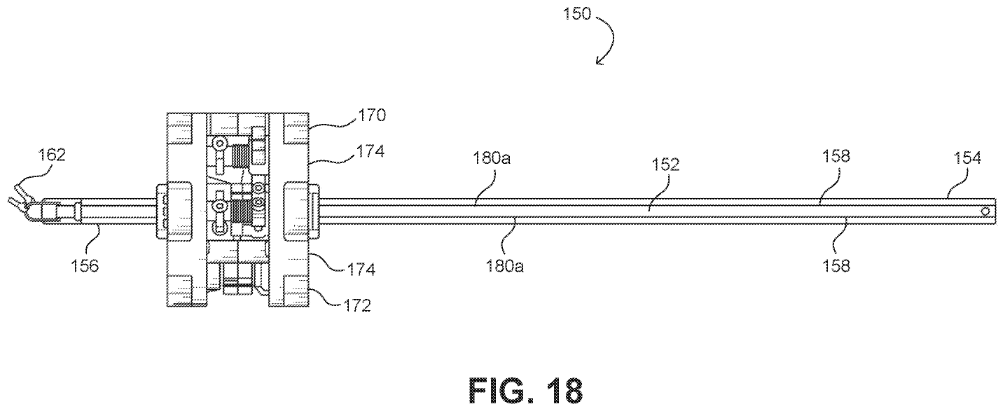

FIG. 18 illustrates an instrument having an instrument based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver.

In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

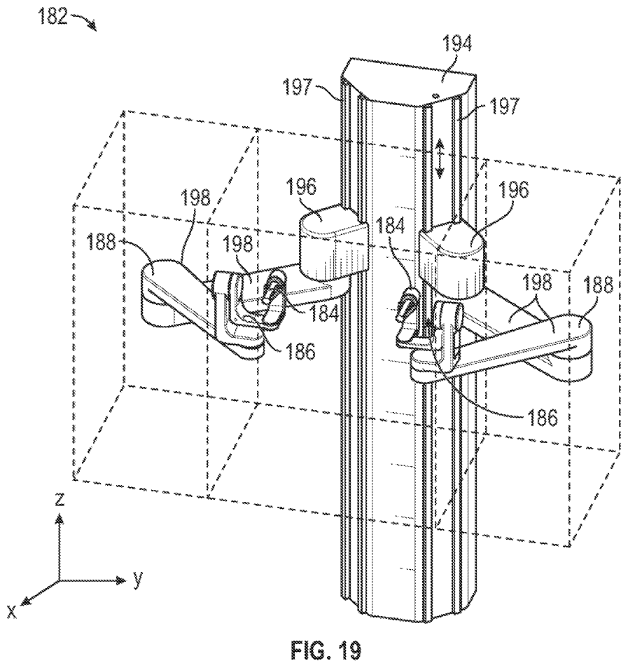

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments, and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

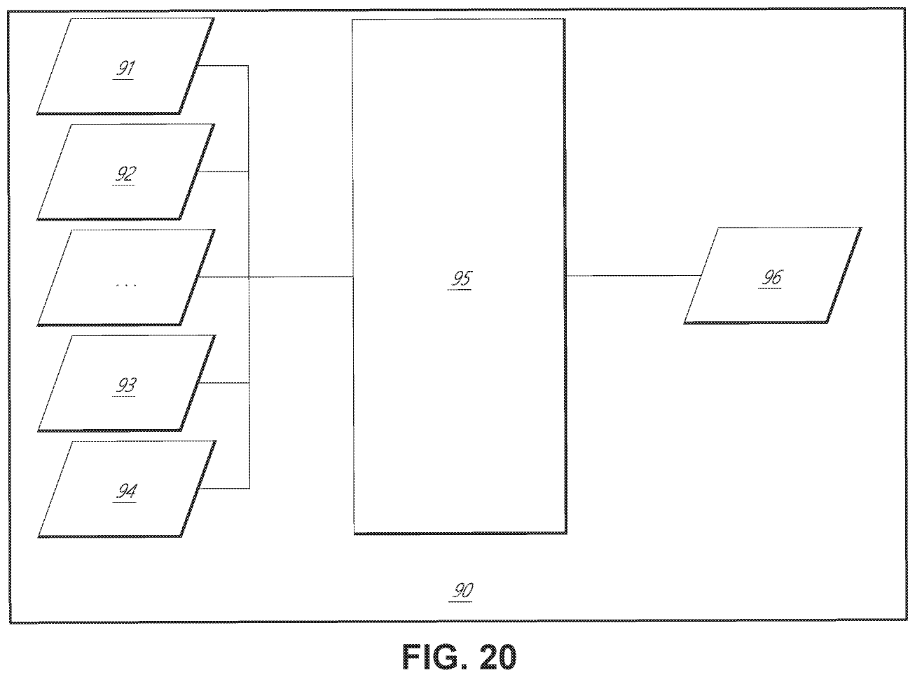

FIG. 20 is a block diagram illustrating a localization system 90 that estimates a location of one or more elements of the robotic system, such as the location of the instrument, in accordance to an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Pre-operative mapping may be accomplished through the use of the collection of low dose CT scans. Pre-operative CT scans are reconstructed into three-dimensional images, which are visualized, e.g., as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images, and are particularly appropriate for bronchoscopy.

In some embodiments, the instrument may be equipped with a camera to provide vision data 92. The localization module 95 may process the vision data to enable one or more vision-based location tracking. For example, the preoperative model data may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intra-operatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising of one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter) may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively “registered” to the patient anatomy (e.g., the preoperative model) in order to determine the geometric transformation that aligns a single location in the coordinate system with a position in the pre-operative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during pre-operative calibration. Intra-operatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Systems, Devices, and Methods for Establishing Procedural Setup

Embodiments of the disclosure relate to systems, methods, and devices for establishing improved procedural setup so that a robotic surgery can be executed smoothly with minimal interruptions.

In accordance with some embodiments of the present disclosure, a robotic medical system comprises a kinematic chain that includes a robotic arm. For example, the kinematic chain can include a robotic arm, or a robotic arm with its underlying bar, or two or more robotic arms and their corresponding bar(s). During setup, the robotic system can adjust (e.g., refine and optimize) at least a portion of the kinematic chain from a first pose (e.g., position and/or orientation) to a second pose based on a procedure selection (e.g., a selection of one or more procedures from a listing of multiple procedures) and either a boundary condition established by the user (e.g., accessory consideration for a selected procedure) or patient information inferred from one or more port locations of the one or more robotic arms.

In accordance with some embodiments of the present disclosure, a robotic system can gather additional information (e.g., from the port locations derived from sensor data detected by the system) after the arms of the robotic system are placed in a docked state. The robotic system further optimizes pose(s) of the robotic arms and/or one or more underlying bars based on the additional information.

In accordance with some embodiments of the present disclosure, a robotic system includes a user interface (e.g., on a tower viewer display, a surgeon viewer display, or a bed pendant with a built-in display, etc.). The user interface displays comparisons (e.g., visual comparisons) between the actual and recommended port location and/or between the actual and recommended bar and/or arm pose to assist user decisions on adjusting bar and/or arm pose(s), optionally, in conjunction with other visual and/or audio feedback, data, instruction, and/or prompts, etc. The user interface can further enable entry and detection of user inputs corresponding to user adjustment or user supervision of a port location, bar pose, and/or arm pose adjustment in response to and in accordance with the displayed comparisons and other visual and/or audio feedback, data, instruction, and/or prompts, in accordance with some embodiments.

### A. Robotic System

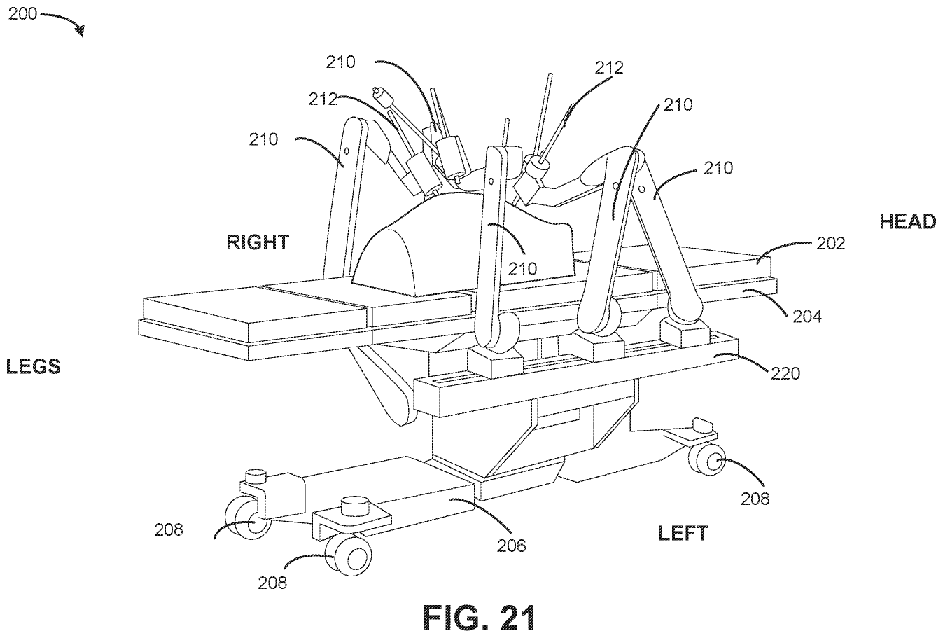

FIG. 21 illustrates an exemplary robotic system 200 according to some embodiments. In some embodiments, the robotic system 200 is a robotic medical system (e.g., robotic surgery system). In the example of FIG. 21, the robotic system 200 comprises a patient support platform 202 (e.g., a patient platform, a table, a bed, etc.). The two ends along the length of the patient support platform 202 are respectively referred to as “head” and “leg”. The two sides of the patient support platform 202 are respectively referred to as “left” and “right.” The patient support platform 202 includes a support 204 (e.g., a rigid frame) for the patient support platform 202.

The robotic system 200 includes a base 206 for supporting the robotic system 200. The base 206 includes wheels 208 that allow the robotic system 200 to be easily movable or repositionable in a physical environment. In some embodiments, the wheels 208 are omitted from the robotic system 200 or are retractable, and the base 206 can rest directly on the ground or floor. In some embodiments, the wheels 208 are replaced with feet.

The robotic system 200 includes one or more robotic arms 210. The robotic arms 210 can be configured to perform robotic medical procedures as described above with reference to FIGS. 1-20. Although FIG. 21 shows five robotic arms 210, it should be appreciated that the robotic system 200 may include any number of robotic arms, including less than five or six or more.

The robotic system 200 also includes one or more bars 220 (e.g., adjustable arm support or an adjustable bar) that support the robotic arms 210. Each of the robotic arms 210 is supported on, and movably coupled to, a bar 220, by a respective base joint of the robotic arm. In some embodiments, and as described in FIG. 12, bar 220 can provide several degrees of freedom, including lift, lateral translation, tilt, etc. In some embodiments, each of the robotic arms 210 and/or the adjustable arm supports 220 is also referred to as a respective kinematic chain.

FIG. 21 shows three robotic arms 210 supported by the bar 220 that is in the field of view of the figure. The two remaining robotic arms are supported by another bar that is located across the other length of the patient support platform 202.

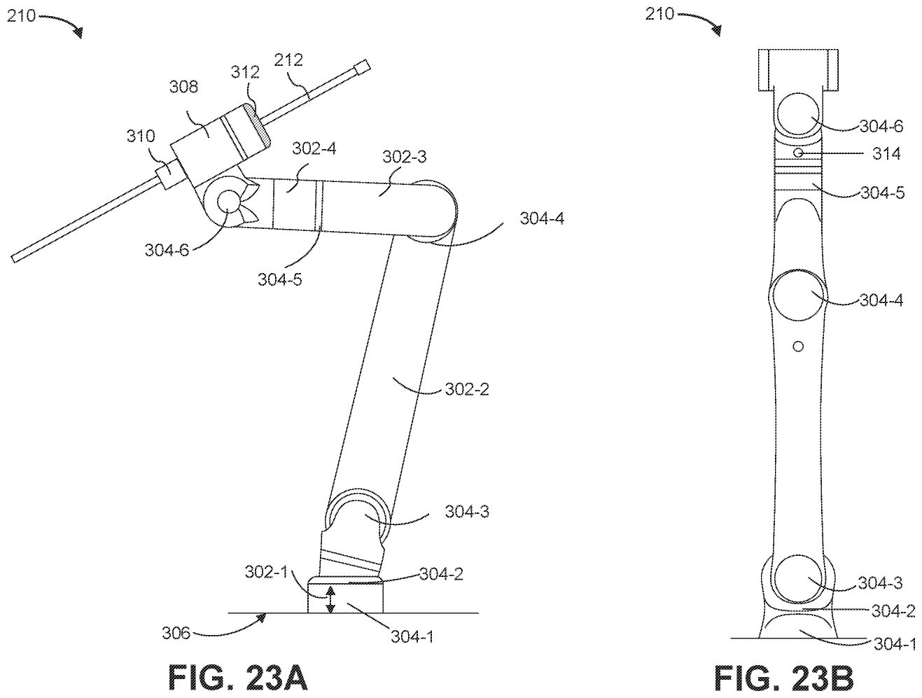

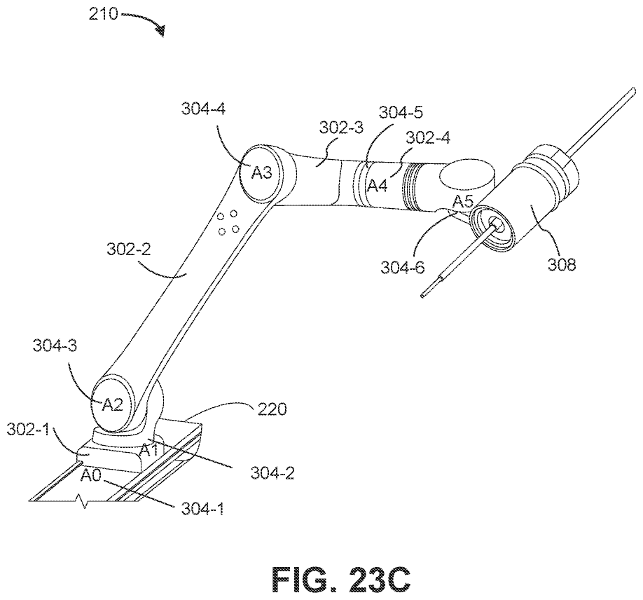

In some embodiments, the adjustable arm supports 220 can be configured to provide a base position for one or more of the robotic arms 210 for a robotic medical procedure. A robotic arm 210 can be positioned relative to the patient support platform 202 by translating the robotic arm 210 along a length of its underlying bar 220 and/or by adjusting a position and/or orientation of the robotic arm 210 via one or more joints and/or links (see, e.g., FIG. 23). In some embodiments, the bar pose can be changed via manual manipulation, teleoperation, and/or power assisted motion.

In some embodiments, the adjustable arm support 220 can be translated along a length of the patient support platform 202. In some embodiments, translation of the bar 220 along a length of the patient support platform 202 causes one or more of the robotic arms 210 supported by the bar 220 to be simultaneously translated with the bar or relative to the bar. In some embodiments, the bar 220 can be translated while keeping one or more of the robotic arms stationary with respect to the base 206 of the robotic medical system 200.

In the example of FIG. 21, the adjustable arm support 220 is located along a partial length of the patient support platform 202. In some embodiments, the adjustable arm support 220 may extend across an entire length of the patient support platform 202, and/or across a partial or full width of the patient support platform 202.

During a robotic medical procedure, one or more of the robotic arms 210 can also be configured to hold instruments 212 (e.g., robotically-controlled medical instruments or tools, such as an endoscope and/or any other instruments (e.g., sensors, light, cutting instrument, etc.) that may be used during surgery), and/or be coupled to one or more accessories, including one or more cannulas, in accordance with some embodiments.

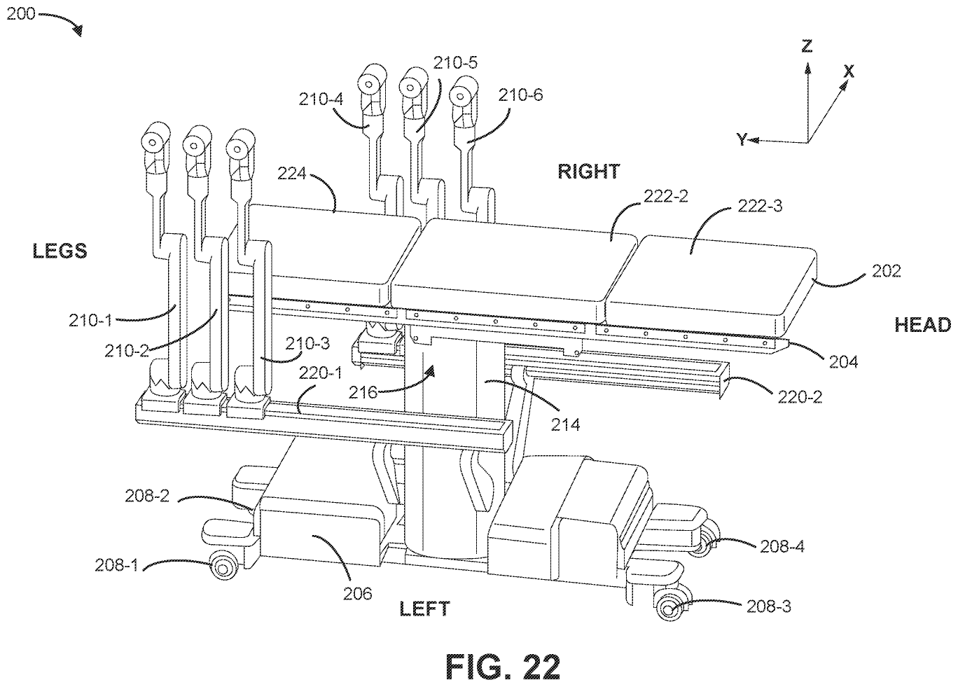

FIG. 22 illustrates another view of the exemplary robotic system 200 in FIG. 21 according to some embodiments. In this example, the robotic medical system 200 includes six robotic arms 210-1, 210-2, 210-3, 210-4, 210-5, and 210-6. The patient platform 202 is supported by a column 214 that extends between the base 206 and the patient platform 202. In some embodiments, the patient platform 202 comprises a tilt mechanism 216. The tilt mechanism 216 can be positioned between the column 214 and the patient platform 202 to allow the patient platform to pivot, rotate, or tilt relative to the column 214. The tilt mechanism 216 can be configured to allow for lateral and/or longitudinal tilt of the patient platform 202. In some embodiments, the tilt mechanism 216 allows for simultaneous lateral and longitudinal tilt of the patient platform 202.

FIG. 22 shows the patient platform 202 in an untilted state or position. In some embodiments, the untilted state or position may be a default position of the patient platform 202. In some embodiments, the default position of the patient platform 202 is a substantially horizontal position as shown. As illustrated, in the untilted state, the patient platform 202 can be positioned horizontally or parallel to a surface that supports the robotic medical system 200 (e.g., the ground or floor). In some embodiments, the term “untilted” may refer to a state where the angle between the default state and the current state is less than a threshold angular amount (e.g., less than 5 degrees, less than an amount that would cause the patient to shift on the patient platform, etc.). In some embodiments, the term “untilted” may refer to a state where the patient platform is substantially perpendicular to the direction of gravity, irrespective of the angle of the surface that supports the robotic medical system relative to gravity.

With continued reference to FIG. 22, in the illustrated example of the robotic system 200, the patient platform 202 includes a support 204. In some embodiments, the support 204 comprises a rigid support structure or frame, and can support one or more surfaces, pads, or cushions 222. An upper surface of the patient platform 202 can comprise a support surface 224. During a medical procedure, a patient can be placed on the support surface 224.

FIG. 22 shows the robotic arms 210 and the adjustable arm supports 220 in an exemplary deployed configuration in which the robotic arms 210 reach above the patient platform 202. In some embodiments, due to the configuration of the robotic system 200, which enables stowage of different components beneath the patient platform 202, the robotic arms 210 and the arm supports 220 can occupy a space underneath the patient platform 202. Thus, in some embodiments, it may be advantageous to configure the tilt mechanism 216 to have a low-profile and/or low volume to maximize the space available for storage below.

FIG. 22 also illustrates an example, x, y, and z coordinate system that may be used to describe certain features of the embodiments disclosed herein. It will be appreciated that this coordinate system is provided for purposes of example and explanation only and that other coordinate systems may be used. In the illustrated example, the x-direction or x-axis extends in a lateral direction across the patient platform 202 when the patient platform 202 is in an untilted state. That is, the x-direction extends across the patient platform 202 from one lateral side (e.g., the right side) to the other lateral side (e.g., the left side) when the patient platform 202 is in an untilted state. The y-direction or y-axis extends in a longitudinal direction along the patient platform 202 when the patient platform 202 is in an untilted state. That is, the y-direction extends along the patient platform 202 from one longitudinal end (e.g., the head end) to the other longitudinal end (e.g., the legs end) when the patient platform 202 is in an untilted state. In an untilted state, the patient platform 202 can lie in or be parallel to the x-y plane, which can be parallel to the floor or ground. In the illustrated example, the z-direction or z-axis extends along the column 214 in a vertical direction. In some embodiments, the tilt mechanism 216 is configured to laterally tilt the patient platform 202 by rotating the patient platform 202 about a lateral tilt axis that is parallel to the y-axis. The tilt mechanism 216 can further be configured to longitudinally tilt the patient platform 202 by rotating the patient platform 202 about a longitudinal tilt axis that is parallel to the x-axis.

### B. Robotic Arm

FIGS. 23A to 23C illustrate different views of an exemplary robotic arm 210 according to some embodiments.

FIG. 23A illustrates that the robotic arm 210 includes a plurality of links 302 (e.g., linkages). The links 302 are connected by one or more joints 304. Each of the joints 304 includes one or more degrees of freedom (DoFs).

In FIG. 23A, the joints 304 include a first joint 304-1 (e.g., a base joint or an A0 joint) that is located at or near a base 306 of the robotic arm 210. In some embodiments, the base joint 304-1 comprises a prismatic joint that allows the robotic arm 210 to translate along the bar 220 (e.g., along the y-axis). The joints 304 also include a second joint 304-2 (e.g., an A1 joint). In some embodiments, the second joint 304-2 rotates with respect to the base joint 304-1. The joints 304 also include a third joint 304-3 (e.g., an A2 joint) that is connected to one end of link 302-2. In some embodiments, the joint 304-3 includes multiple DoFs and facilitates both tilt and rotation of the link 302-2 tilt with respect to the joint 304-3.

FIG. 23A also shows a fourth joint 304-4 (e.g., an A3 joint) that is connected to the other end of the link 302-2. In some embodiments, the joint 304-4 comprises an elbow joint that connects the link 302-2 and the link 302-3. The joints 304 further comprise a pair of joints 304-5 (e.g., a wrist roll joint or an A4 joint) and 304-6 (e.g., a wrist pitch joint or an A5 joint), which is located on a distal portion of the robotic arm 210.

A proximal end of the robotic arm 210 may be connected to a base 306 and a distal end of the robotic arm 210 may be connected to an advanced device manipulator (ADM) 308 (e.g., a tool driver, an instrument driver, or a robotic end effector, etc.). The ADM 308 may be configured to control the positioning and manipulation of a medical instrument 212 (e.g., a tool, a scope, etc.).

The robotic arm 210 can also include a cannula sensor 310 for detecting presence or proximity of a cannula to the robotic arm 210. In some embodiments, the robotic arm 210 is placed in a docked state (e.g., docked position) when the cannula sensor 310 detects presence of a cannula (e.g., via one or more processors of the robotic system 200). In some embodiments, when the robotic arm 210 is in a docked position, the robotic arm 210 can execute null space motion to maintain a position and/or orientation of the cannula, as discussed in further detail below. Conversely, when no cannula is detected by the cannula sensor 310, the robotic arm 210 is placed in an undocked state (e.g., undocked position).

In some embodiments, and as illustrated in FIG. 23A, the robotic arm 210 includes an input or button 312 (e.g., a donut-shaped button, or other types of controls, etc.) that can be used to place the robotic arm 210 in an admittance mode (e.g., by depressing the button 312). The admittance mode is also referred to as an admittance scheme or admittance control. In the admittance mode, the robotic system 210 measures forces and/or torques (e.g., imparted on the robotic arm 210) and outputs corresponding velocities and/or positions. In some embodiments, the robotic arm 210 can be manually manipulated by a user (e.g., during a set-up procedure, or in between procedures, etc.) in the admittance mode. In some instances, by using admittance control, the operator need not overcome all of the inertia in the robotic system 200 to move the robotic arm 210. For example, under admittance control, when the operator imparts a force on the arm, the robotic system 200 can measure the force and assist the operator in moving the robotic arm 210 by driving one or more motors associated with the robotic arm 210, thereby resulting in desired velocities and/or positions of the robotic arm 210.

In some embodiments, the links 302 may be detachably coupled to the medical tool 212 (e.g., to facilitate ease of mounting and dismounting of the medical tool 212 from the robotic arm 210). The joints 304 provide the robotic arm 210 with a plurality of degrees of freedom (DoFs) that facilitate control of the medical tool 212 via the ADM 308.

FIG. 23B illustrates a front view of the robotic arm 210. FIG. 23C illustrates a perspective view of the robotic arm 210. In some embodiments, the robotic arm 210 includes a second input or button 314 (e.g., a push button) that is distinct from the button 312 in FIG. 23A, for placing the robotic arm 210 in an impedance mode (e.g., by a single press or continuous press and hold of the button 314). In this example, the button 314 is located between the A4 joint 304-5 and the A5 joint 304-6. The impedance mode is also referred to as impedance scheme or impedance control. In the impedance mode, the robotic system 200 measures displacements (e.g., changes in position and velocity) and outputs forces to facilitate manual movement of the robotic arm. In some embodiments, the robotic arm 210 can be manually manipulated by a user (e.g., during a set-up procedure) in the impedance mode. In some embodiments, under the impedance mode, the operator's movement of one part of a robotic arm 210 may back drive other parts of the robotic arm 210.

In some embodiments, for admittance control, a force sensor or load cell can measure the force that the operator is applying to the robotic arm 210 and move the robotic arm 210 in a way that feels light. Admittance control may feel lighter than impedance control because, under admittance control, one can hide the perceived inertia of the robotic arm 210 because motors in the controller can help to accelerate the mass. In contrast, with impedance control, the user is responsible for most if not all mass acceleration, in accordance with some embodiments.

In some circumstances, depending on the position of the robotic arm 210 relative to the operator, it may be inconvenient to reach the button 312 and/or the button 314 to activate a manual manipulating mode (e.g., the admittance mode and/or the impedance mode). Accordingly, under these circumstances, it may be convenient for the operator to trigger the manual manipulation mode other than by buttons.

In some embodiments, the robotic arm 210 comprises a single button that can be used to place the robotic arm 210 in the admittance mode and the impedance mode (e.g., by using different presses, such as a long press, a short press, press and hold etc.). In some embodiments, the robotic arm 210 can be placed in impedance mode by a user pushing on arm linkages (e.g., the links 302) and/or joints (e.g., the joints 304) and overcoming a force threshold.

During a medical procedure, it can be desirable to have the ADM 308 of the robotic arm 210 and/or a remote center of motion (RCM) of the tool 212 coupled thereto kept in a static pose (e.g., position and/or orientation). An RCM may refer to a point in space where a cannula or other access port through which a medical tool 212 is inserted is constrained in motion. In some embodiments, the medical tool 212 includes an end effector that is inserted through an incision or natural orifice of a patient while maintaining the RCM. In some embodiments, the medical tool 212 includes an end effector that is in a retracted state during a setup process of the robotic medical system.

In some circumstances, the robotic system 200 can be configured to move one or more links 302 of the robotic arm 210 within a “null space” to avoid collisions with nearby objects (e.g., other robotic arms), while the ADM 308 of the robotic arm 210 and/or the RCM are maintained in their respective poses (e.g., positions and/or orientations). The null space can be viewed as the space in which a robotic arm 210 can move that does not result in movement of the ADM 308 and/or RCM, thereby maintaining the position and/or the orientation of the medical tool 212 (e.g., within a patient). In some embodiments, a robotic arm 210 can have multiple positions and/or configurations available for each pose of the ADM 308.

For a robotic arm 210 to move the ADM 308 to a desired pose in space, in certain embodiments, the robotic arm 210 may have at least six DoFs—three DoFs for translation (e.g., X, Y, and Z positions) and three DoFs for rotation (e.g., yaw, pitch, and roll). In some embodiments, each joint 304 may provide the robotic arm 210 with a single DoF, and thus, the robotic arm 210 may have at least six joints to achieve freedom of motion to position the ADM 308 at any pose in space. To further maintain the ADM 308 of the robotic arm 210 and/or the remote center or motion in a desired pose, the robotic arm 210 may further have at least one additional “redundant joint.” Thus, in certain embodiments, the system may include a robotic arm 210 having at least seven joints 304, providing the robotic arm 210 with at least seven DoFs. In some embodiments, the robotic arm 210 may include a subset of joints 304 each having more than one degree of freedom thereby achieving the additional DoFs for null space motion. However, depending on the embodiment, the robotic arm 210 may have a greater or fewer number of DoFs.

Furthermore, as described in FIG. 12, the bar 220 (e.g., adjustable arm support) can provide several degrees of freedom, including lift, lateral translation, tilt, etc. Thus, depending on the embodiment, a robotic medical system can have many more robotically controlled degrees of freedom beyond just those in the robotic arms 210 to provide for null space movement and collision avoidance. In a respective embodiment of these embodiments, the end effectors of one or more robotic arms (and any tools or instruments coupled thereto) and a remote center along the axis of the tool can advantageously maintain in pose and/or position within a patient.

A robotic arm 210 having at least one redundant DoF has at least one more DoF than the minimum number of DoFs for performing a given task. For example, a robotic arm 210 can have at least seven DoFs, where one of the joints 304 of the robotic arm 210 can be considered a redundant joint, in accordance with some embodiments. The one or more redundant joints can allow the robotic arm 210 to move in a null space to both maintain the pose of the ADM 308 and a position of an RCM and avoid collision(s) with other robotic arms or objects.

In some embodiments, the robotic system 200 can be configured to perform collision avoidance to avoid collision(s), e.g., between adjacent robotic arms 210, by taking advantage of the movement of one or more redundant joints in a null space. For example, when a robotic arm 210 collides with or approaches (e.g., within a defined distance of) another robotic arm 210, one or more processors of the robotic system 200 can be configured to detect the collision or impending collision (e.g., via kinematics). Accordingly, the robotic system 200 can control one or both of the robotic arms 210 to adjust their respective joints within the null space to avoid the collision or impending collision. In an embodiment including at least a pair of robotic arms, a base of one of the robotic arms and its end effector can stay in its pose, while links or joints therebetween move in a null space to avoid collisions with an adjacent robotic arm.

### C. Setup Workflow

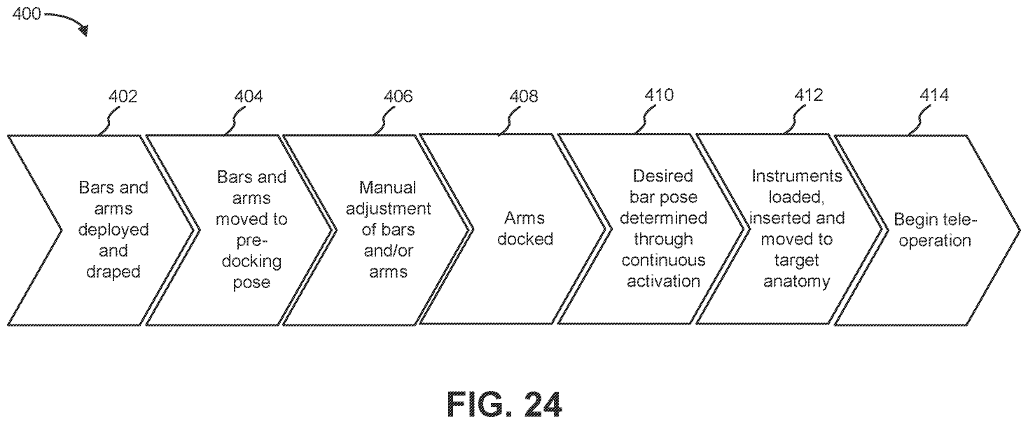

FIG. 24 illustrates a setup workflow 400 for a robotic system 200 in accordance with some embodiments.

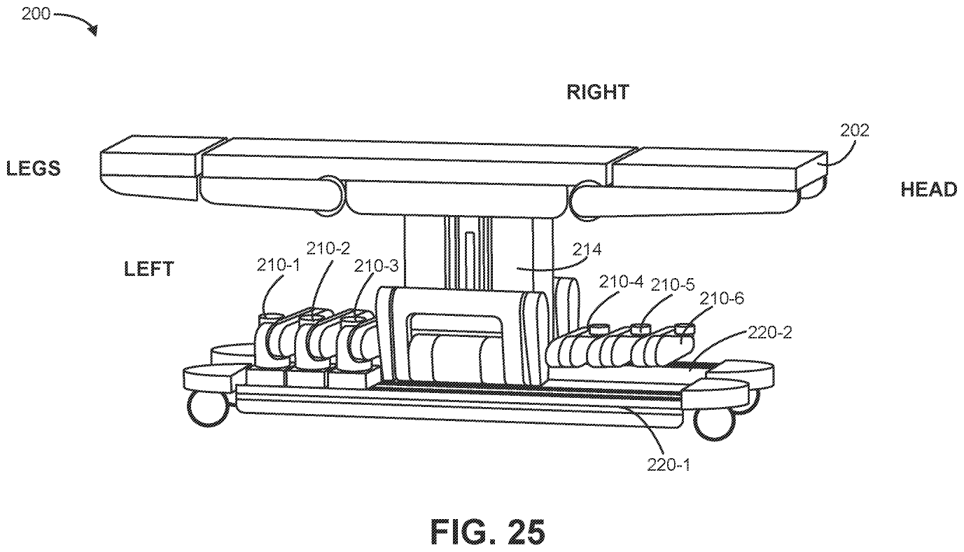

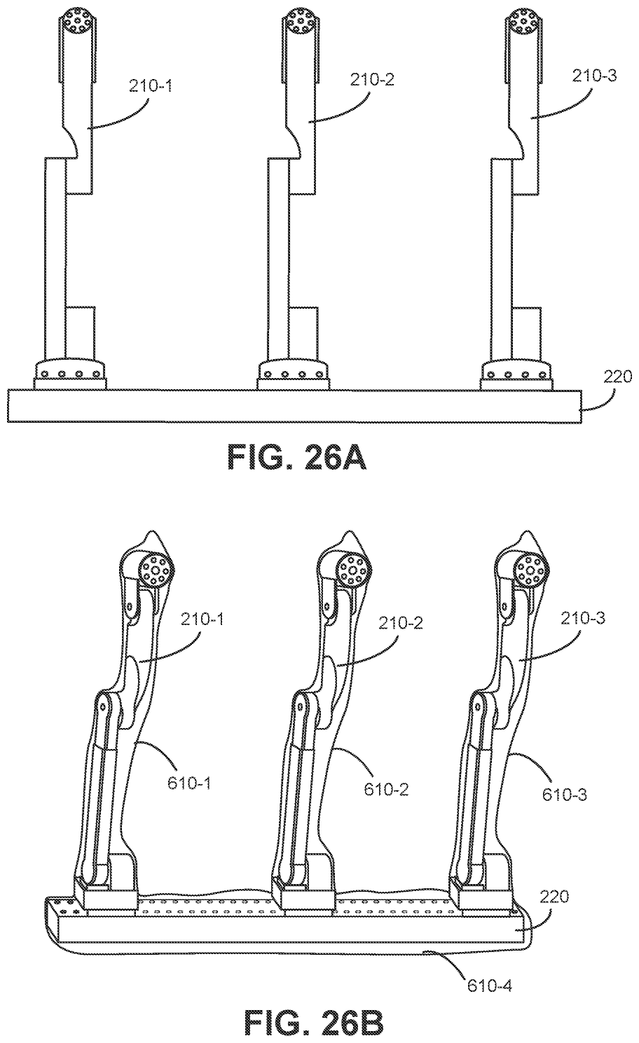

The workflow 400 includes, in step 402, deploying the bars 220 and the robotic arms 210 from a stowed state to a deployed state. FIG. 25 illustrates a robotic system 200 in a stowed state in accordance with some embodiments. In FIG. 25, the robotic arms 210 and the bars 220 are stowed beneath the table 202 of the robotic system 200. FIG. 26A illustrates three robotic arms 210 and their underlying bar 220 in an initial deployed state, in accordance with some embodiments. In some embodiments, the robotic arms 210 and the bar 220 can be draped when they are in the initial deployed state. FIG. 26B depicts the robotic arms 210 and the bar 220 covered by drapes 610 (e.g., surgical drapes, surgical covering, etc.), to isolate the robotic arms 210 and the bar 220 from a patient's body during surgery, in accordance with some embodiments.

With continued reference to FIG. 24, in step 404, the robotic arms 210 and the bars 220 are moved to a pre-docking pose after they have been draped. In some embodiments, the robotic system (e.g., via one or more processors) executes movement to robotically move the arms 210 and/or bars 220 to the pre-docking pose. In some embodiments, the robotic system executes movement to robotically move the arms 210 and/or bars 220 to the pre-docking pose in accordance with a recommended pose that corresponds to a surgical procedure to be performed via the robotic system.

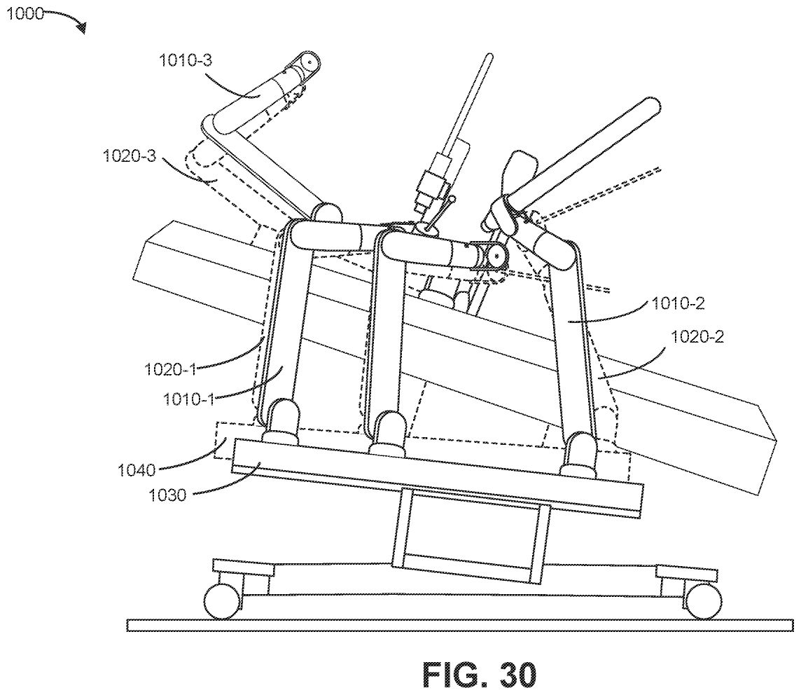

In some embodiments, the robotic movement of the bars 220 and/or arms 210 to a particular pre-docking pose is in response to user (e.g., a surgeon assistant, patient side staff, surgeon, etc.) selection of a particular procedure on a user interface (e.g., a user interface 1000, FIG. 30) located on a tower display or a bed pendant of the robotic system. For example, in some embodiments, different surgical procedures have different bar and arm setups (e.g., different pre-docking setups, each of the setups having a corresponding bar-and-arm-pose configuration) that ease the subsequent docking of the robotic arms in step 408. The user interface can include one or more interface elements that identify the surgical procedures. User selection of a procedure via a corresponding interface element causes the robotic system to automatically cause (e.g., execute) movement (e.g., robotic movement) to move the arms 220 and bars 210 to a particular pre-docking pose corresponding to the selected procedure. In some embodiments, the one or more processors cause (e.g., execute) robotic movement of the arms 210 and/or bars 220 to the pre-docking pose in accordance with a current state of the patient.

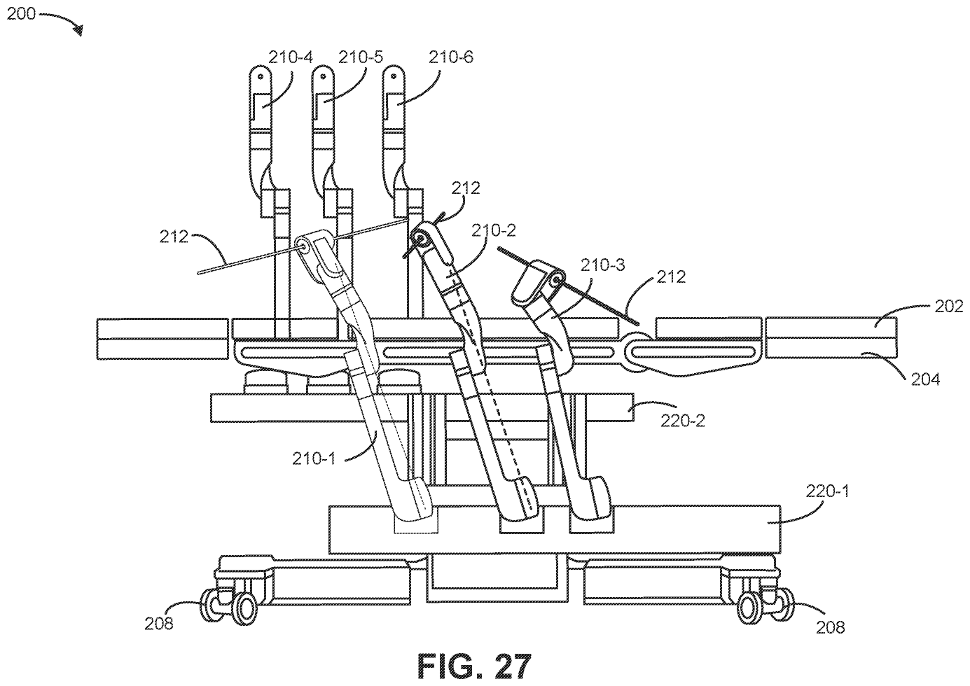

FIG. 27 illustrates a robotic system 200 that includes one set of robotic arms (e.g., robotic arms 210-4, 210-5, and 210-6) in the background that are in a pre-docking pose, in accordance with some embodiments. FIG. 27 also shows another set of robotic arms (e.g., robotic arms 210-1, 210-2, and 210-3) in the foreground that are being robotically moved to a pre-docking pose, in accordance with some embodiments. FIG. 27 also illustrates that in some embodiments, after the foreground robotic arms (e.g., robotic arms 210-1, 210-2, and 210-3) have been moved to the pre-docking pose, they are docked and loaded with instruments 212. The proactive adjustments described herein are, optionally, performed before loading of the instruments, in some embodiments. The view of the patient as well as the respective ports/cannulas within the patient have been excluded from FIG. 27 in order to enhance the visibility of the robotic arms 210.

With continued reference to FIG. 24, in some embodiments, the workflow 400 includes, in step 406, manual adjustment (e.g., manual movement) of the bars 220 and/or arms 210. Examples of manual adjustment include manual robotic arm jogging and manual bar jogging (e.g., bar pose jogging). In some embodiments, manual robotic arm jogging can be performed via admittance control (e.g., by activating admittance mode in the robotic arm as described with respect to FIG. 23A). For example, in some embodiments, manual robotic arm jogging can be performed by pressing an input control or button, such as the button 312 in FIG. 23A. In some embodiments, manual bar jogging can be performed via a user actuating a tower or bed pendant.

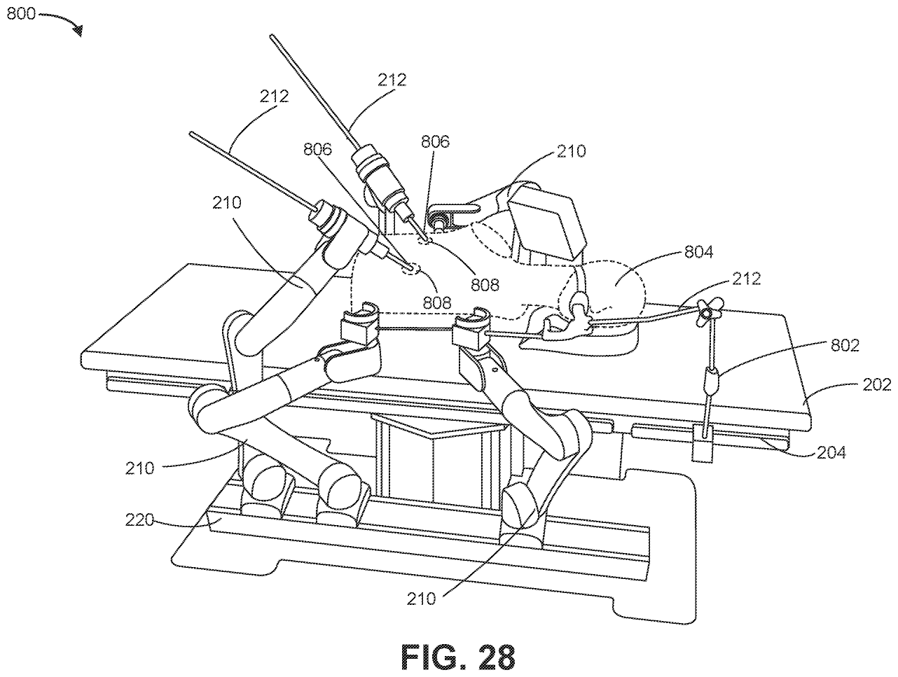

In some embodiments, step 406 refers to manual bar and/or arm adjustment by a user, whereby the user establishes boundary conditions for safety reasons. The boundary conditions are unique to a particular surgery and can also be based on an accessory that is utilized for the procedure, and/or based on patient information. FIG. 28 provides an example of boundary condition establishment.

FIG. 28 illustrates an exemplary setup 800 for a surgery (e.g., a combined endoscopic thoracoscopic surgery, or CETS) that is performed using a robotic system, in accordance with some embodiments. The setup 800 includes an accessory 802 that is connected to a support 204 (e.g., a rigid frame, or a bed rail) of the robotic system. Using FIG. 28 as an example, in some embodiments, a user can establish a boundary condition by taking into consideration the accessory 802 and establishing (e.g., ascertaining, determining, etc.) how close the bar 220 can be brought to the table-top 202 in view of the accessory 802. In such a scenario, the user can manually move the bar 220 to a position that is close to the bed 202 while taking into consideration the location of the accessory 802. In some embodiments, the manual adjustment of the bar 220 to a particular position in turn informs the robotic system that the position of the bar 220 is safely within bounds, thereby allowing the robotic system to establish a boundary condition.

In some embodiments, a boundary condition can be established based on patient information (e.g., information of a patient 804), such as patient fixation (e.g., how the patient 804 is fixed to or positioned on the bed 202), patient size, etc.

In some embodiments, the sequence in which step 404 and step 406 are executed is interchangeable. That is to say, in some embodiments, step 404 can be executed before step 406. In some embodiments, step 404 can be executed after step 406. In some embodiments, step 404 and step 406 can be executed as an iterative loop in which the steps of robotic movements (step 404) and manual movements (step 406) are iterated successively. In some embodiments, the step 404 and the step 406 collectively form a single pre-docking setup step, in which the bars 220 and the robotic arms 210 can be moved robotically as well as manually, and the order of execution of the robotic and manual movements is flexible.

With continued reference to FIG. 24, in some embodiments, the workflow 400 includes, in step 408, placing the robotic arms 210 in a docked state. In some embodiments, the robotic arms 210 are docked to one or more cannulas whereby they can be subsequently coupled to one or more corresponding instruments 212 (e.g., robotically-controlled medical instruments or tools, such as an endoscope, a laparoscope, and/or any another instruments that may be used during surgery) in step 412. In some embodiments, the robotic arms 210 are individually docked via admittance docking. For example, a user will handle the robotic arm 210 (e.g., via the button 312 in FIG. 23A) using one hand and handle a cannula using the other hand. The user will then bring the robotic arm 210 to the cannula, thereby placing the robotic arm 210 in a docked state.

FIG. 28 depicts the robotic arms docked to cannulas in accordance with some embodiments. In some embodiments, with the robotic arms 210 docked to the corresponding cannulas, the robotic system 200 determines a remote center of motion (RCM) 806 and/or a port of entry 808 with respect to each of the robotic arms 210. In other words, placing the robotic arms 210 in a docked state facilitates the determination of the respective RCM 806 and/or port of entry 808. As used herein, an RCM 806 may refer to a point in space where a cannula or other access port through which a medical tool/instrument is inserted is constrained in motion, or a pivot point along an axis of the instrument. In some embodiments, the RCM 806 refers to a point of intersection of a cannula and the patient's body. In some embodiments, the RCM 806 has a corresponding port of entry 808 that is located on the body of a patient 804. The port of entry 808 is an entry point on the patient 804 for the robotic arm 210 (e.g., robotic manipulator). In some embodiments, the port of entry 808 (or, e.g., port, port location, entry point, port region, port area, port position, etc.) refers to the position on the patient's body through which the medical tool/instrument is inserted and constrained in motion. In some embodiments, the port of entry 808 corresponds to an incision point (or an incision region) that is made through the skin of the patient 804 to facilitate a medical operation or procedure. In some embodiments, the port of entry 808 corresponds to a natural orifice, such as a mouth of the patient 804 (e.g., for a bronchoscopy procedure). In some embodiments, when a port of entry 808 is known to (e.g., established or determined by) the robotic system 200, the robotic system 200 can further infer other information, such as patient size, clinical techniques, etc.

FIG. 24 also illustrates that in some embodiments, the workflow 400 includes, in step 410, determining a desired bar and/or arm pose through continuous activation. In some embodiments, even when the robotic arms 210 are in a docked state, there may still be some additional adjustments to the bar 220 and/or arms 210 that may be beneficial prior to starting a surgical procedure. For example, in some circumstances, even when a robotic arm 210 is docked to a respective cannula, one may still want to adjust the port location (e.g., port of entry 808) corresponding to the robotic arm 210. One example of such an adjustment, in accordance with some embodiments, is “port tenting.” In port tenting, a port of entry and/or a cannula corresponding to the port of entry can be robotically or manually adjusted (e.g., by a few millimeters) by taking advantage of the elasticity of the patient.

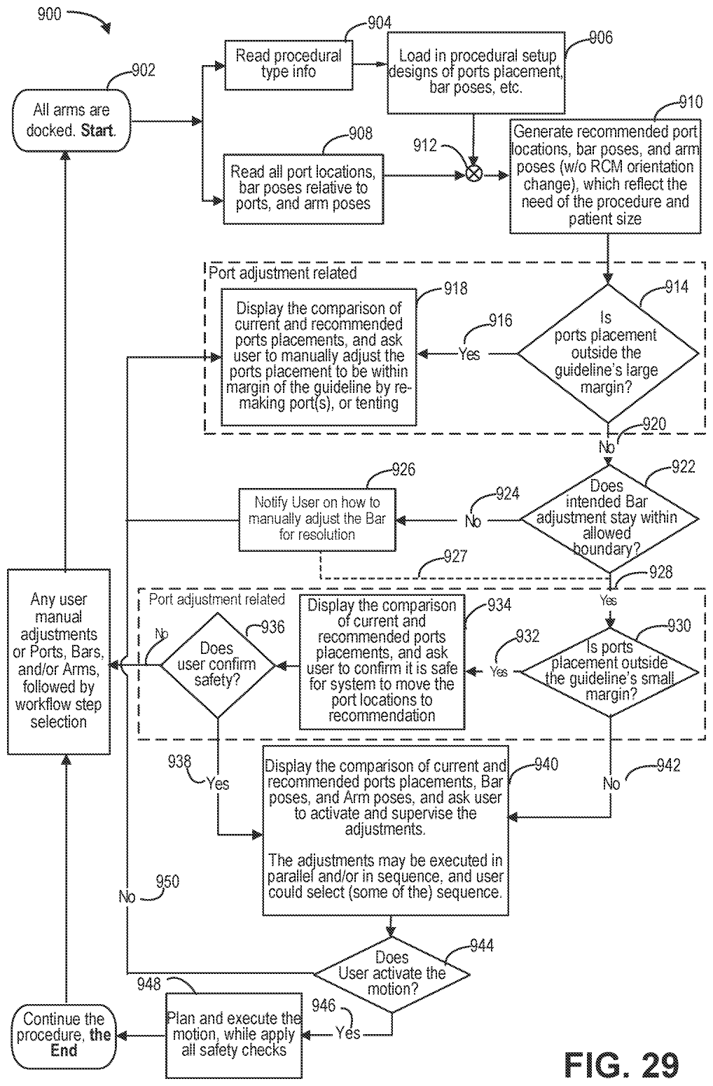

In some embodiments, in accordance with the adjustment to the port location and/or cannula position, the robotic system 200 determines and establishes a new RCM corresponding to the adjusted port location. As the additional information corresponding to the new RCM is being generated, the robotic system 200 adjusts a pose (e.g., position and/or orientation) of an underlying bar 220 and associated robotic arm(s) 210 according to the new RCM. In other words, the robotic system 200 advantageously determines a desired bar and/or arm pose from both pre-set procedural selections (e.g., as described with respect to steps 404 and 406) as well as from information that is being generated as a port location is being adjusted. Consequently, a bar 220 and/or one or more of its associated robotic arms 210 are being optimized based on additional information that has been generated from the continuous movement of the port(s) in step 410. The advantage is that such additional information can be unique based on the location of accessories, patient size, etc. Accordingly, the bar and/or arm pose is optimized based on additional information that is unique to a particular patient. Further details for determining a desired bar and/or arm pose through continuous activation are described in FIG. 29.

In some embodiments, the robotic system 200 is capable of resolving any conflict between a recommended bar and/or arm pose (e.g., a pre-docking pose that is generated in step 404 via user selection of a procedure) and an actual bar and/or arm pose, while taking into consideration boundary conditions. In other words, the robotic system 200 can leverage the recommended bar and/or arm pose to refine a generated trajectory for user-supervised bar and//or arm motion, while still complying with constraints such as a bar boundary condition, or a pose of a robotic arm end effector etc.

With continued reference to FIG. 24, in some embodiments, after the bars 220 and/or the robotic arms 210 have been moved to their desired poses according to step 410, the instruments can then be loaded, inserted, and moved to a target anatomy of the patient, as illustrated in step 412. In other embodiments, step 412 (e.g., instrument loading) can be performed before step 410 (e.g., adjustment to desired poses). In step 414, the surgeon can begin teleoperation.

In some embodiments, the steps 402, 404, 406, 408, 410, and 412 comprise a “pre-operation phase” and the step 414 is referred to as an “intra-operation phase.” The pre-operation phase includes setting up the robotic system, positioning the robotic arms, underlying bars, accessories etc. with respect a patient, and then introducing the instruments, in accordance with some embodiments. The “intra-operation phase” corresponds when a surgeon commences an operation to when the operation is completed in accordance with some embodiments.

FIG. 29 illustrates a flow diagram 900 for determining a desired bar pose through continuous activation (e.g., step 410 of FIG. 24) in accordance with some embodiments. In some embodiments, the processes described in the flow diagram 900 are executed by one or more processors (e.g., in FIG. 20) of a robotic medical system (e.g., the robotic system 200 as illustrated in FIGS. 21, 22, 26, 27, and 28) in accordance with instructions stored on memory of the robotic medical system.

In some embodiments, the processors determine (902) that the robotic arms (e.g., robotic arms 210 in FIGS. 21, 22, 23A, 23B, 23C, and 27) are in a docked state. In accordance with a determination that the robotic arms 210 are in a docked state, the processors read (904) procedural type information corresponding to a procedural selection. In some embodiments, the procedural selection comprises a procedural that is selected by a user of the robotic system in accordance with step 404 of FIG. 24. In accordance with the procedural selection, the processors load (906) procedural setup designs of cannula positions, ports placement, bar poses, arm poses, etc., corresponding to the selected procedure.

In some embodiments, in accordance with a determination that the robotic arms 210 are in a docked state, the processors read (908) (e.g., determine, ascertain, etc.) the actual port location(s), the actual bar pose(s) (e.g., actual bar poses relative to the ports), and/or the actual arm pose(s) of the robotic system. In some embodiments, the actual port location(s) can be determined via the kinematics of a robotic arm 210 corresponding to the port of entry. For example, in some embodiments, a robotic arm 210 includes joint encoder(s) that are positioned on the robotic arm, which measure positions and/or angles of the joints of the robotic arm.

With continued reference to FIG. 29, in some embodiments, the processors generate (910) recommended port locations, and generate one or more recommended bar and/or arm poses (e.g., locations and/or poses without RCM orientation change) in accordance with a comparison (912) between the procedural setup designs, the actual port locations and the actual bar and/or arm poses. In some embodiments, after the processors generate a recommended port location, the processors compare the recommended port location the actual port location. In some embodiments, comparing the actual port location with the recommended port location includes determining a separation distance between the actual port location and the recommended port location.

In some embodiments, the processors determine (914) whether the separation distance is within a “large margin.” The large margin can represent a distance that one can safely apply stress to a patient. For example, the large margin may be a pre-set distance having a range of approximately 1 mm to 20 mm, or more approximately 3 mm to 10 mm, in accordance with some embodiments. In some embodiments, in accordance with a determination that the actual port placement is beyond (916) the proscribed large margin (e.g., beyond the pre-set distance), the processors will generate and display (918) (e.g., visually and/or audibly) a comparison of the current and recommended port placements. For example, in some embodiments, the processors display the comparison visually via a user interface on a tower viewer or surgeon viewer of the robotic system, such as the user interface 1000 in FIG. 30. In some embodiments, the processors will also generate and display (918) a notification requesting that the user manually adjust (e.g., manually move, reposition etc.) the port placements to be within the large margin, such as by re-making the port of entry or by port tenting.

In some embodiments, in accordance with a determination that the separation distance between the actual port location and the recommended port location is within (920) (e.g., less than or equal to, not beyond, does not exceed, etc.) the large margin, the processors compare the recommended bar and/or arm pose with the actual bar and/or arm pose. For example, the processors determine whether an intended bar and/or arm adjustment (e.g., an adjustment from the actual bar pose to the recommended bar pose, or an adjustment from the actual arm pose to the recommended arm pose) is (922) within an allowed boundary. In some embodiments, the allowed boundary corresponds to a boundary condition that has been established by a user (e.g., for safety reasons) in accordance with step 406 of FIG. 24. For example, as discussed with respect to FIGS. 24 and 28, the boundary condition can be established based a position of an accessory, patient fixation, and/or other patient information, etc. It is to be noted that the actual bar and/or arm poses will always be within the allowed boundary because as described in FIG. 24, the arms are docked in their positions (step 408) only after the boundary conditions have been established (step 406).

In some embodiments, in accordance with a determination that the intended bar and/or arm adjustment does not (924) stay within the allowed boundary (e.g., exceeds the allowed boundary, is exactly at the limit of the allowed boundary, etc.), the processors notify (926) the user on how to manually adjust the bar and/or arm pose. In some instances, the user may decide to continue with the procedure without manually adjusting the bar and/or arm pose. In this scenario, later robotic bar and/or arm adjustments will approach the recommended bar and/or arm pose as much as the boundary condition allows (e.g., as indicated by the dotted line path 927). However, the later robotic adjustments will not reach the recommended bar and/or arm pose.

In some embodiments, in accordance with a determination that the intended bar and/or arm adjustment exceeds the allowed boundary, the one or more processors generate and execute a modified target pose that meets the boundary condition.

In some embodiments, in accordance with a determination that the intended bar and/or arm adjustment is (928) within the allowed boundary, the processors compare (930) the separation distance between the actual port location and the recommended port location with a pre-determined distance or a predetermined “small margin.” The small margin has a smaller separation distance than the large margin. In some embodiments, while the large margin is concerned with safety (e.g., the large margin which represents a distance that one can safely apply stress to a patient), the small margin has to do with accuracy. For example, any adjustment within the small margin is guaranteed to be safe; the goal is to bring the actual port location closer to the recommended port location so as minimize disruptions during the tele-operative surgery. In some embodiments, the small margin represents a distance that inquires whether “the actual port location is close enough to the recommended port location.” In some embodiments, the small margin can be from e.g., 0 mm and 15 mm, or more approximately from 0 mm to 6 mm.

Referring again to FIG. 29, in some embodiments, in accordance with a determination that the separation distance between the actual port location and the recommended port location is beyond (932) the small margin (e.g., exceeds the small margin, larger than the small margin etc.), the processors generate and display (934) a comparison between the current and recommended port placements. The processors also generate and display (934) a notification requesting user confirmation that it is safe for the processors to move the port location from the actual port location to the recommended port location. The processors also determine (936) whether the user has confirmed safety. In some embodiments, in accordance with a determination that the user has confirmed safety (938), the processors display (940) the comparison of the current and recommended port placements, the comparison of current and recommended bar pose, and/or current and recommended arm pose. The processors also display (940) a notification requesting the user to activate and supervise the adjustments.

In some embodiments, the comparison(s) are displayed as visual feedback (e.g., visualizations) in a user interface of the robotic system. FIG. 30 illustrates an exemplary user interface 1000 depicting a visualization that shows (e.g., compares) the actual arm poses 1010 (e.g., as solid lines) and the recommended arm poses 1020 (e.g., as dotted lines), and the actual bar pose 1030 (e.g., as solid lines) and the recommended bar pose 1040 (e.g., as dotted lines) in accordance with some embodiments.

In some embodiments, the user interface 1000 also displays a visualization that shows (e.g., compares) the actual port location and the first recommended port location.

In some embodiments, the processors also generate and display, in the user interface 1000, one or more projected trajectories showing how the processors would execute movement to robotically move from the actual poses to the recommended poses. In some embodiments, the user interface 1000 is displayed on a tower viewer, surgeon viewer, and/or pendant of the robotic system.

In some embodiments, the user interface 1000 also displays one or more interface elements that, when selected by the user, causes the one or more processors to automatically execute movement (e.g., robotic movement) to move the bar and/or arm pose from the actual bar and/or arm pose to the recommended bar and/or arm pose.

In some embodiments, the user interface 1000 also displays one or more interface elements that, when selected by the user, causes the one or more processors to automatically execute movement (e.g., robotic movement) to move the port location from the actual port location to the recommended port location.

Referring back to FIG. 29, in some embodiments, in accordance with a determination (944) that the user has activated (946) the adjustment (e.g., the processors receive a user command to activate the adjustment), the processors plan (948) and execute the motion while applying all safety checks. In some embodiments, in accordance with a determination (944) that the user has not activated (950) the adjustment (e.g., a timeout for receiving the user command has been reached), the processors will generate and display (918) a comparison of the current and recommended port placements, and a notification requesting that the user manually adjust (e.g., manually move, reposition etc.) the port placements to be within the margins of the guideline.

### D. Exemplary Processes for Procedural Setup

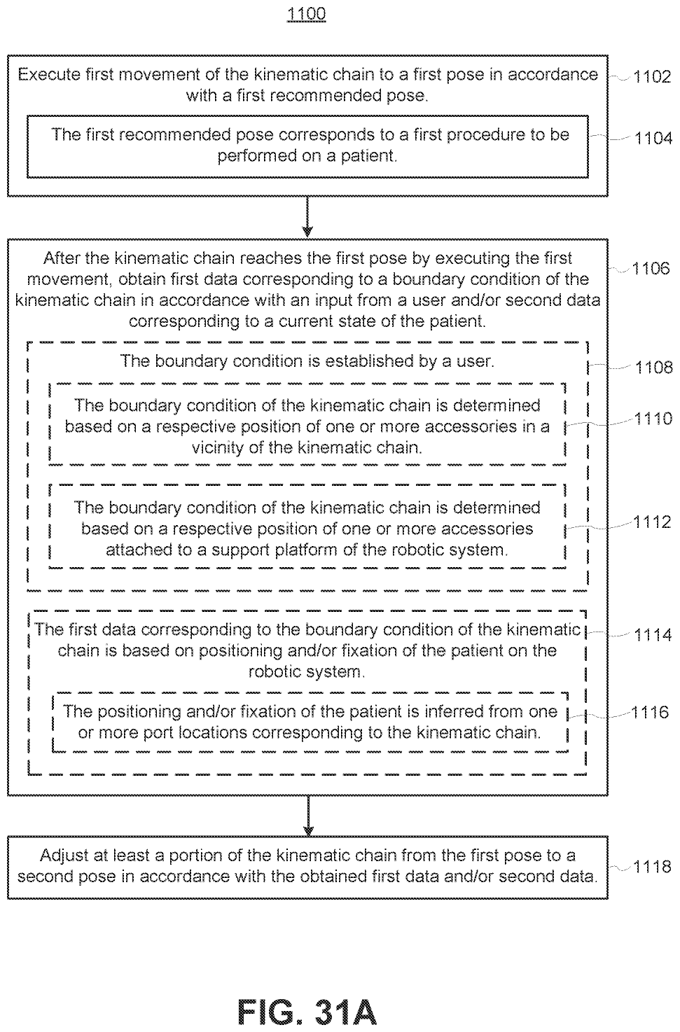

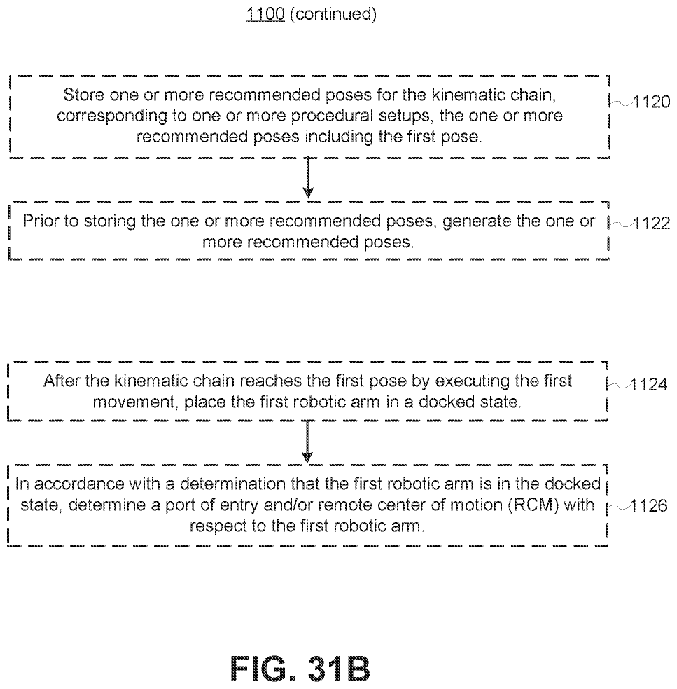

FIGS. 31A and 31B illustrate a flowchart diagram for a method 1100 for procedural setup performed by one or more processors of a robotic system (e.g., the robotic medical system 200 as illustrated in FIGS. 21 and 22, or a robotic surgery platform, etc.), in accordance with some embodiments.

The robotic system comprises a kinematic chain that includes at least a first robotic arm (e.g., a robotic manipulator) (e.g., the robotic arm 210 in FIGS. 21, 22, 23A, 23B, 23C, 25, 26A, 26B, 27, and 28). In some embodiments, the kinematic chain includes an adjustable bar (e.g., bar 220, FIGS. 21, 22, 25, 26A, 26B, 27, and 28) connected to one or more robotic arms, or a robotic arm coupled to an adjustable bar, or two or more robotic arms coupled to an adjustable bar, etc.

The robotic system executes (1102) (e.g., causes) first movement of the kinematic chain to a first pose in accordance with a first recommended pose.

In some embodiments, the first recommended pose corresponds (1104) to a first procedure to be performed on a patient.

For example, in some embodiments, the first movement of the kinematic chain includes translation and/or rotation of at least a link or a joint of the kinematic chain relative to the physical environment or a patient platform of the robotic system. In some embodiments, the first movement of the kinematic chain comprises movement of only the underlying bar (e.g., the robotic arm is kept stationary during movement of the underlying bar), movement of one or more robotic arms without movement of the bar, or movement of both the bar and one or more robotic arms, etc.

In some embodiments, the first recommended pose comprises a first pre-docking pose that is pre-stored in the robotic system. In some embodiments, the first recommended pose comprises a dynamically generated pose based on user input, or a pose that is generated based on stored model pose and customized by user manual manipulation or teleoperation, etc.

In some embodiments, the first movement of the kinematic chain to the first recommended pose enables the kinematic chain (e.g., robotic arms) to dock to their respective ports. In some embodiments, the first movement of the kinematic chain is triggered after a user (e.g., a surgeon or an assistant) selects a first procedure (e.g., a first procedural setup) corresponding to the first recommended pose (e.g., on a user interface, such as a user interface on a tower viewer of robotic system, a user console etc.). The user input can be interpreted by the processors as selection of the first recommended pose. In some embodiments, the processors control movement of the kinematic chain. For example, the kinematic chain is moved robotically to the first recommended pose, as described with respect to step 404 in FIG. 24. In some embodiments, movement of the kinematic chain includes manual adjustment by a surgeon assistant or patient side staff, such as for safety, as described with respect to step 406 in FIG. 24. Examples of manual adjustment can include manual robotic arm and bar jogging. In some embodiments, manual robotic arm jogging can be performed via admittance control (e.g., pressing an input or a button on the robotic arm, as described with respect to FIG. 23). In some embodiments, manual bar jogging can be performed via a user actuating a tower or bed pendant of the robotic system. In some embodiments, the first recommended pose is stored in the memory of the robotic system. In some embodiments, the first recommended pose can be generated on the fly or retrieved from a remote server.

Referring again to FIGS. 31A-31B, in some embodiments, after the kinematic chain reaches the first pose by executing the first movement, the robotic system obtains (1106) first data corresponding to a boundary condition of the kinematic chain in accordance with an input from a user and/or second data corresponding to a current state of the patient. For example, in some embodiments, the second data include data on patient size, patient position, and/or patient posture, etc. In some embodiments, the second data can be derived from sensor data or data specifying port locations of the robotic arms, etc. In some embodiments, prior to obtaining the first data and the second data, the robotic system places the robotic arms in a docked state, as described in step 408 of FIG. 24.

In some embodiments, the boundary condition of the kinematic chain is established (1108) by a user.

In some embodiments, the boundary condition of the kinematic chain is determined (1110) based on a respective position of one or more accessories in a vicinity of the kinematic chain.

For example, in some embodiments, the kinematic chain includes set-up joints (including an adjustable bar) and the robotic arm. The one or more accessories can include, for example, a uterine manipulator that is attached to a rail of the bed that is in the vicinity of the kinematic chain. Other accessories can include, for example, stirrups, arm extenders, or other accessories that may be coupled to the bed to assist in surgery.

In some embodiments, the user can help to establish boundary conditions that are unique to a particular surgery. An example of a boundary condition can include, for example, a user taking into a consideration an accessory (e.g., an arm extender accessory) that is connected to the bed rail (see, e.g., FIG. 28) and establishing how close the bar can be brought to the table-top in view of the accessory. In such a scenario, the user can manually move the bar to a position close to the bed while taking into consideration the location of the accessory. This informs the system that the position of the bar is safely within bounds, thereby allowing the system to establish a boundary condition.

In some embodiments, the boundary condition of the kinematic chain is determined (1112) based on a respective position of one or more accessories attached to a support platform (e.g., a bed, a patient platform, a patient support platform, a table etc.) of the robotic system.

In some embodiments, the first data corresponding to the boundary condition of the kinematic chain is based (1114) on positioning and/or fixation of the patient on the robotic system (e.g., on a support platform of the robotic system). In some embodiments, rather than having a boundary condition be based on a location of an accessory, the boundary condition can be based on patient information, including patient fixation (e.g., how the patient is fixed to the bed).

In some embodiments, the positioning and/or fixation of the patient is inferred (1116) from one or more port locations corresponding to the kinematic chain. In some embodiments, a port location refers to an entry point (e.g., port of entry 808, FIG. 28) on the patient for a robotic arm of the kinematic chain. In some embodiments, a port location refers to a location on a patient's body through which the medical tool/instrument is inserted and constrained in motion. The port location has a corresponding point along an axis of a cannula of the robotic arm. The intersection between the port location and the corresponding point is the remote center of motion (RCM) of the robotic arm.

The robotic system adjusts (1118) at least a portion of the kinematic chain from the first pose to a second pose in accordance with the obtained first data and/or second data.

In some embodiments, the robotic system adjusts at least a portion of the kinematic chain by executing one or more additional movements to optimize and/or refine a position and/or orientation of at least a portion of the kinematic chain, such as a position and/or orientation of a cannula on a robotic arm of the kinematic chain, or a link of the kinematic chain, or a link of a robotic arm (e.g., a link connected to a joint, a link between two joints of the robotic arm, etc.), or a joint of a robotic arm, or a position of the underlying bar, etc.

In some embodiments, adjusting at least a portion of the kinematic chain includes adjusting a link and/or joint of a robotic arm, such as the first robotic arm. In some embodiments, adjusting at least a portion of the kinematic chain includes adjusting a position, rotation, and/or tilt of an underlying bar of a robotic arm (e.g., the arm is kept stationary while adjusting the bar). In some embodiments, adjusting at least a portion of the kinematic chain includes adjusting both a link and/or joint of the robotic arm as well as a position, rotation, and/or tilt etc. of the underlying bar.

In some embodiments, the robotic system stores (1120) one or more recommended poses for the kinematic chain, corresponding to one or more procedural setups. The one or more recommended poses include the first pose.

In some embodiments, the stored recommended poses comprise one or more pre-docking poses for the kinematic chain, corresponding to one or more procedures to be executed by the kinematic chain. In some embodiments, the stored recommended poses include a position and/or orientation of a robotic arm of the kinematic chain, a position and/or orientation of a cannula of a robotic arm of the kinematic chain, and/or a position and/or orientation of an adjustable bar of the kinematic chain, etc.

In some embodiments, the one or more procedural setups correspond to one or more procedures to be executed by the robotic system (e.g., via the kinematic chain). Each of the recommended poses reflects the need of the procedure and takes into consideration the patient size, patient position, and/or accessory setup, etc. As an example, procedures can include invasive procedures, minimally invasive procedures, and/or non-invasive procedures etc. Examples of minimally invasive procedure include laparoscopy or combined endoscopic thoracoscopic surgery (CETS). An example of a non-invasive procedure includes endoscopy. Endoscopic procedures can further include bronchoscopy, ureteroscopy, gastroscopy, etc.

In some embodiments, the robotic system can generate the one or more recommended poses on the fly. In some embodiments, the robotic system can retrieve the one or more recommended poses from a remote server.

With continued reference to FIGS. 31A-31B, in some embodiments, prior to storing the one or more recommended poses, the robotic system generates (1122) the one or more recommended poses.

For example, in some embodiments, the robotic system generates the one or more recommended poses based on the designs of a procedural setup. In some embodiments, the designs of the procedural setup can come from procedural development and are coded into the software, including port locations and their relative relationship (e.g., with a robotic arm, a bar, patient bed etc.), bar poses relative to port locations, arm poses such as preferred initial A0 joint positions on the bar, etc. The designs of the procedural setup can be based on heuristics, predefined rules, trained model(s) from supervised and/or unsupervised learning, and/or a combination of different methods on various parts of the designs in accordance with some embodiments. The designs can adapt to available information obtained at the procedure time, including information corresponding to procedure type, surgeon preference, and/or patient size information that are inferred from the port locations read by the system after all the robotic arms of the system are docked. In some embodiments, the recommended poses can be generated on the fly or retrieved from a remote server.

In some embodiments, after the kinematic chain reaches the first pose by executing the first movement, the robotic system places (1124) the first robotic arm in a docked state (e.g., a docked position).

For example, in some embodiments, the first robotic arm can include a cannula sensor for detecting presence or proximity of a cannula to the first robotic arm. The first robotic arm is placed in a docked state (e.g., a docked position) when the cannula sensor detects presence of a cannula (e.g., via the one or more processors). In some embodiments, when the first robotic arm is in a docked position, the first robotic arm can execute null space motion to maintain a pose (e.g., a position and/or orientation of the cannula).

In some embodiments, the robotic system includes a plurality of robotic arms. In some embodiments, each of the robotic arms is individually docked via admittance docking. For example, a user will handle the robotic arm 210 (e.g., via the button 312 in FIG. 23A) using one hand and handle a cannula using the other hand. The user will then bring the robotic arm 210 to the cannula, thereby placing the robotic arm 210 in a docked state.

In some embodiments, in accordance with a determination that the first robotic arm is in the docked state, the robotic system determines (1126) a port of entry (e.g., port of entry 808, FIG. 28) and/or remote center of motion (RCM) (e.g., RCM 806, FIG. 28) with respect to the first robotic arm.

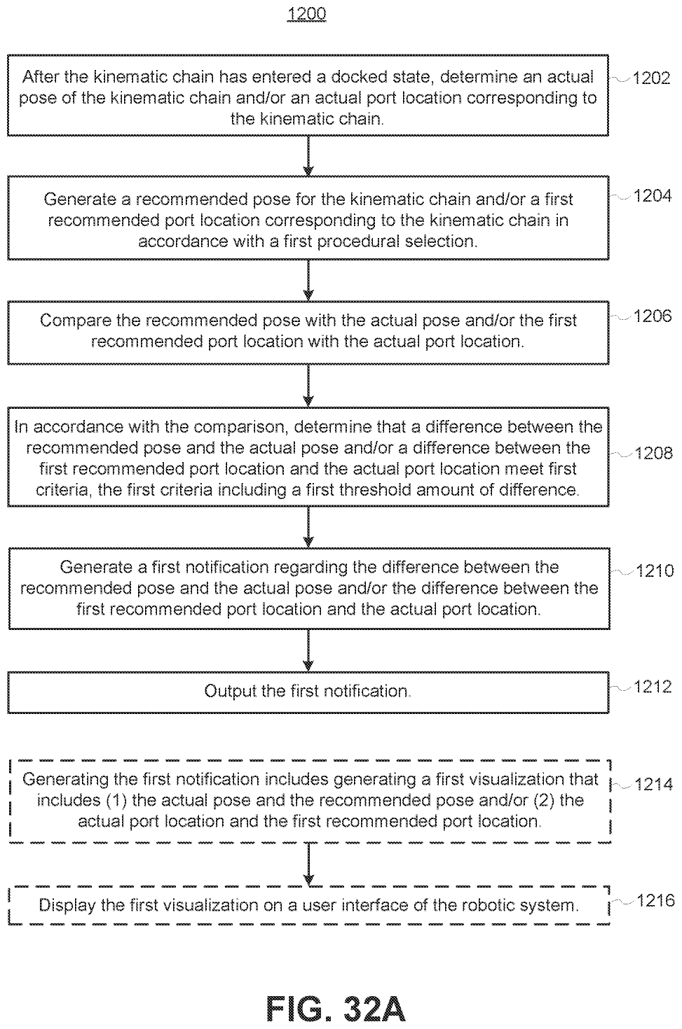

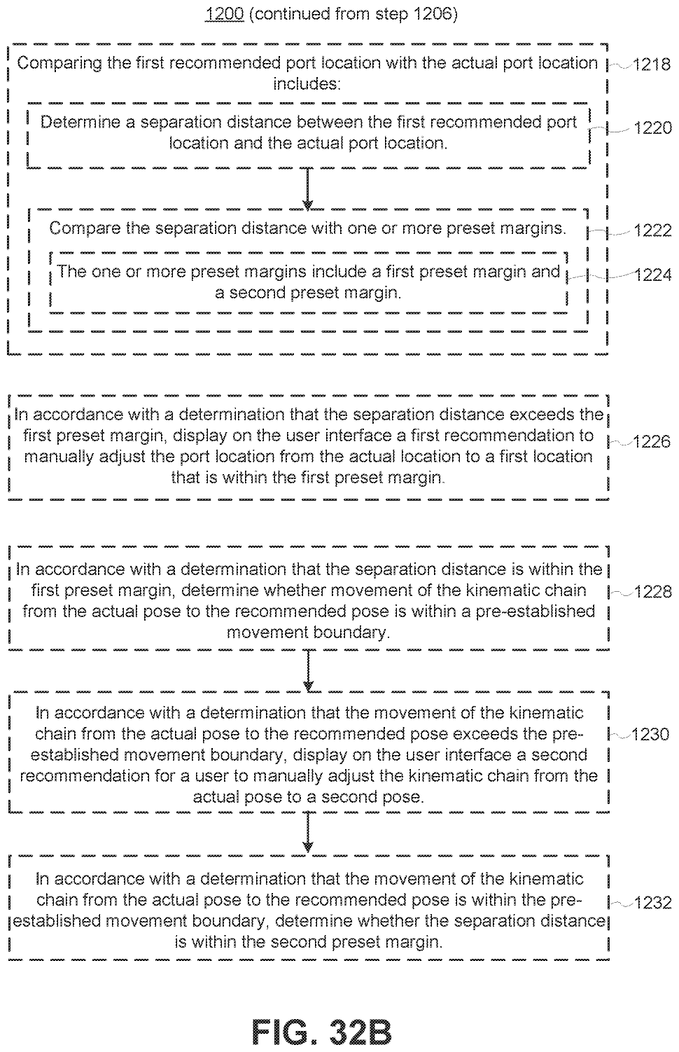

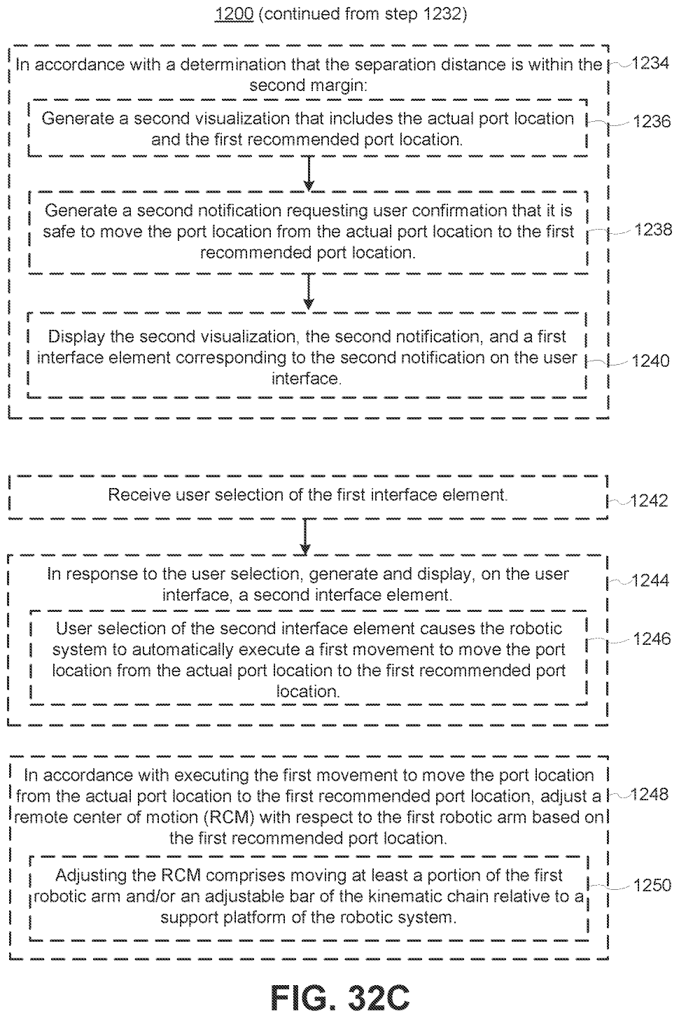

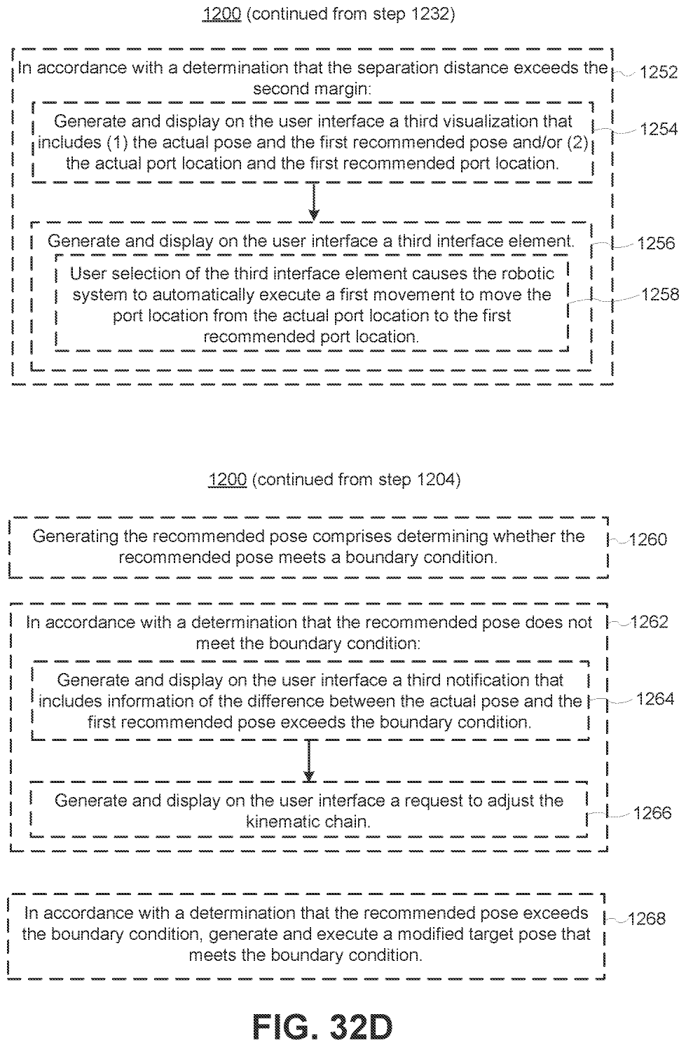

FIGS. 32A to 32D illustrate a flowchart diagram for a method 1200 for determining bars and/or arm poses performed by one or more processors of a robotic system (e.g., the robotic medical system 200 as illustrated in FIGS. 21 and 22, or a robotic surgery platform, etc.), in accordance with some embodiments.

The robotic system comprises a display (e.g., display on a tower viewer or a bed pendant of the robotic system). The robotic system also comprises a kinematic chain. For example, in some embodiments, the kinematic chain comprises a robotic arm, an adjustable bar, a robotic arm coupled to an adjustable bar, or two or more robotic arms coupled to an adjustable bar, etc.

In accordance with some embodiments, after the kinematic chain has entered a docked state, the robotic system determines (1202) an actual pose of the kinematic chain and/or an actual port location corresponding to the kinematic chain. This is illustrated in step 908 in FIG. 29.

In some embodiments, the robotic system determines an actual port location via the kinematics of a robotic arm of the kinematic chain. In some embodiments, the kinematic chain includes two or more robotic arms and the robotic system determines an actual port location for each of the robotic arms of the kinematic chain. In some embodiments, a robotic arm includes joint encoder(s) that are positioned on the robotic arm, which measure positions and/or angles of the joints of the robotic arm. The robotic system determines the actual position of the robotic arm according to the joint encoder data.

The robotic system generates (1204) a recommended pose for the kinematic chain and/or a first recommended port location corresponding to the kinematic chain in accordance with a first procedural selection. For example, this is illustrated in step 910 of FIG. 29.

In some embodiments, the recommended pose for the kinematic chain includes a recommended position and/or orientation of a robotic arm of the kinematic chain, or a recommended position and/or orientation of a cannula of a robotic arm of the kinematic chain, or a recommended position and/or orientation of an underlying bar of the kinematic chain, etc.

In some embodiments, the recommended pose for the kinematic chain corresponds to a first procedure to be executed by the robotic system (e.g., via the kinematic chain). In some embodiments, the recommended pose reflects the need of the procedure and takes into consideration the patient size, patient position, and/or accessory setup, etc.

In some embodiments, the first procedural selection is selected by a user of the robotic system.

In some embodiments, the kinematic chain includes a robotic arm (e.g., a robotic manipulator) and a port location (e.g., port of entry 808, FIG. 28) is an entry point on the patient for the robotic arm. A port location (or a port, a port of entry, a point, a region etc.) is a location on a patient's body through which the medical tool/instrument is inserted and constrained in motion. The port location (on a patient's body) has a corresponding point along an axis of a cannula of the robotic arm. The intersection between the port location and the corresponding point is the remote center of motion (RCM) of the robotic arm.

In some embodiments, the first recommended pose and/or the first recommended port location are generated in accordance with a first procedural selection and in accordance with the actual pose of the kinematic chain and/or the actual port location of the kinematic chain.

The robotic system compares (1206) the recommended pose with the actual pose and/or the first recommended port location with the actual port location.

In accordance with the comparison, the robotic system determines (1208) that a difference (e.g., a mismatch, distinction) between the recommended pose and the actual pose and/or a difference (e.g., a mismatch, distinction) between the first recommended port location and the actual port location meet first criteria. The first criteria include a first threshold amount of difference. In some embodiments, the first criteria include one or more predefined thresholds, such as thresholds corresponding to position(s), orientation(s), distance(s), and/or relative angle(s), etc. In some embodiments, the first criteria are based on a respective position of one or more accessories for the procedure, on patient positioning and/or fixation of the robotic system etc. In some embodiments, the first criteria are based a small margin for ports placement, and/or a large margin for port placement, etc.

The robotic system generates (1210) a first notification regarding the difference between the recommended pose and the actual pose and/or the difference between the first recommended port location and the actual port location.

The robotic system outputs (1212) the first notification. For example, in some embodiments, the robotic system displays the first notification on a user interface (e.g., user interface 1000, FIG. 30). The user interface is displayed on a tower viewer, a surgeon viewer, a bed pendant, etc. of the robotic system. In some embodiments, the robotic system outputs an audio alert, or a prompt via an audio output device of the robotic system, etc.

In some embodiments, the kinematic chain includes a first robotic arm that is in the docked state.

In some embodiments, generating the first notification includes generating (1214) a first visualization that includes (1) the actual pose and the recommended pose and/or (2) the actual port location and the first recommended port location. In some embodiments, the robotic system displays (1216) the first visualization on a user interface of the robotic system.

For example, in FIG. 30, the one or more processors of the robotic system generates a visualization showing (e.g., comparing) the actual arm poses 1010 and the recommended arm poses 1020. The visualization of FIG. 30 also shows (e.g., compares) the actual bar pose 1030 and the recommended bar pose 1040. The robotic system displays the visualization on a user interface 1000. In some embodiments, the robotic system generates and displays on the user interface 1000 a second visualization showing (e.g., comparing) the first recommended port location and the actual port location. In some embodiments, the current poses (and/or port locations) are displayed in a color that is different from a color of the recommended poses (and/or port locations).

In some embodiments, the robotic system displays (e.g., on the user interface 1000) a projected trajectory (e.g., a simulated trajectory) showing how the pose will transition from the current pose (port location) to the recommended pose.

In some embodiments, the robotic system displays (e.g., on the user interface) a projected trajectory (e.g., a simulated trajectory) showing how the port location will transition from the current port location to the recommended port location.

In some embodiments, comparing the first recommended port location with the actual port location includes (1218): determining (1220) a separation distance between the first recommended port location and the actual port location, and comparing (1222) the separation distance with one or more preset (e.g., predetermined) margins.

In some embodiments, the one or more preset margins include (1224) a first preset margin (e.g., large margin) and a second preset margin (e.g., small margin).

In some embodiments, the first preset margin corresponds to a larger margin of two or more preset margins. The second preset margin corresponds to a smaller margin of two or more preset margins.

In some embodiments, the first preset margin (e.g., large margin) represents a distance in which one can safely apply stress to a patient. For example, the first preset margin is a pre-set distance having a range of approximately, e.g., 1 mm to 20 mm, or more approximately 3 mm to 10 mm, in accordance with some embodiments.

In some embodiments, the second preset margin (e.g., small margin) represents a distance that inquires whether the actual location of the port is close enough to the recommended port location. For example, in some embodiments, the second preset margin is a pre-set distance having a range from, e.g., 0 mm to 15 mm, or more approximately 0 mm to 6 mm, and that is smaller than the range corresponding to the first preset margin (e.g., large margin). In some embodiments, the large margin is concerned with safety (e.g., anything within the large margin is safe) whereas the small margin has to do with accuracy (e.g., any adjustment within the small margin is guaranteed to be safe. The idea is to bring the actual port location closer to the recommended port location so as minimize disruptions during the tele-operative surgery.

In some embodiments, the first preset margin (e.g., a large margin) is between 3 mm and 10 millimeters (e.g., 3 mm to 10 mm inclusive, greater than 3 mm and less than 10 mm, greater than 3 mm and less than or equal to 10 mm, from 3 mm to less than 10 mm, etc.).

In some embodiments, the second preset margin (e.g., a small margin) is between 0 and 6 millimeters (e.g., 0 mm to 6 mm inclusive, greater than 0 mm and less than 6 mm, greater than 0 mm and less than or equal to 6 mm, from 0 mm to less than 6 mm, etc.).

Referring again to FIGS. 32A-32D, in some embodiments, in accordance with a determination that the separation distance exceeds (e.g., is outside of, larger than, beyond, etc.) the first preset margin (e.g., large margin), the robotic system displays (1226) on a user interface a first recommendation to manually adjust a port location from the actual location to a first location that is within the first preset margin. This is also illustrated in steps 914, 916, and 918 in FIG. 29.

For example, in some embodiments, if the actual port placement is beyond the proscribed first preset margin (e.g., large margin), the robotic system provides a display (e.g., on the tower viewer or surgeon viewer) comparing the current and recommended port placements, thereby giving a user an opportunity to manually change the port placements to be within the margin by re-making the port(s) or via port-tenting. In some embodiments, the comparison comprises a visualization showing the current port placement and the recommended port placement. In some embodiments, the user interface also displays guidelines for moving the port location from the actual location to the first location.

In some embodiments, in accordance with a determination (1228) that the separation distance is within (e.g., does not exceed, less than or equal to) the first preset margin (e.g., large margin), the robotic system determines whether movement of the kinematic chain from the actual pose to the recommended pose is within a pre-established movement boundary. This is illustrated in step 922 in FIG. 29. For example, in some embodiments, the movement of the kinematic chain from the actual pose to the recommended pose comprises an intended bar (and/or arm) adjustment that stays within the pre-established movement boundary. In some embodiments, the pre-established boundary condition is based on a respective position of one or more accessories for the procedure, or based on patient positioning, or based on fixation of the robotic system etc.

In some embodiments, in accordance with a determination (1230) that the movement of the kinematic chain from the actual pose to the recommended pose exceeds the pre-established movement boundary (e.g., step 924, FIG. 29), the robotic system displays on a user interface a second recommendation for a user to manually adjust the kinematic chain from the actual pose to a second pose. In some embodiments, the second pose is a pose that is within the movement boundary. The second pose will not reach the first recommended pose, but will approach it as much as the boundary condition allows.

In some embodiments, in accordance with a determination (1232) that the movement of the kinematic chain from the actual pose to the recommended pose is within the pre-established movement boundary (e.g., step 928, FIG. 29), the robotic system determines whether the separation distance is within the second preset margin (e.g., small margin).

In some embodiments, in accordance with a determination (1234) that the separation distance is within the second preset margin (e.g., step 942, FIG. 29), the robotic system generates (1236) a second visualization that includes the actual port location and the first recommended port location. In some embodiments, the second visualization presents different views (e.g., a perspective view, a top view, a front view, and/or a side view, etc.) to enable to user to look at the recommended movements from different perspectives. The robotic system generates (1238) a second notification requesting user confirmation that it is safe to move a port location from the actual port location to the first recommended port location. That is to say, the second notification gives a user a chance to confirm that it is safe to adjust the port location closer to the recommended position. In some embodiments, the robotic system displays (1240) the second visualization, the second notification, and a first interface element corresponding to the second notification on the user interface. In some embodiments, the user confirms safety by selecting the first interface element.

In some embodiments, the robotic system receives (1242) user selection of the first interface element. In some embodiments, in response to the user selection, the robotic system generates (1244) and displays, on the user interface, a second interface element. For example, in some embodiments, the second user interface element comprises a request to the user to activate and supervise the adjustments.

In some embodiments, user selection (1246) of the second interface element causes the robotic system to automatically execute a first movement to move the port location from the actual port location to the first recommended port location. For example, in some embodiments, the first movement comprises an adjustment of the port location via “port tenting,” wherein a port/cannula can be robotically or manually moved (e.g., by a few mm) by leveraging on the elasticity of a patient's body (e.g., ability of the patient to be stretched, compressed, etc.).

In some embodiments, the kinematic chain comprises a first robotic arm that is in the docked state. In accordance with executing the first movement to move the port location from the actual port location to the first recommended port location, the robotic system adjusts (1248) a remote center of motion (RCM) with respect to the first robotic arm based on the first recommended port location. For example, when the port location changes, the robotic system determines and establishes a new RCM at the new ports. As this additional information is being generated, the surgical system is capable of adjusting the bar and associated robotic arms.

In some embodiments, adjusting the RCM comprises moving (1250) at least a portion of the first robotic arm and/or an adjustable bar of the kinematic chain relative to a support platform of the robotic system. Accordingly, as the additional information of the new RCM is being generated, the robotic system is capable of adjusting the bar and associated robotic arm(s) to further optimize the setup.

In some embodiments, in accordance with a determination (1252) that the separation distance exceeds (e.g., outside of, beyond, larger than) the second margin (e.g., small margin) (e.g., steps 930 and 932, FIG. 29), the robotic system generates (1254) and displays on the user interface a third visualization that includes (1) the actual pose and the first recommended pose and/or (2) the actual port location and the first recommended port location.

In some embodiments, the robotic system also generates (1256) and displays on the user interface a third interface element. In some embodiments, the third interface element requests the user to confirm that it is safe for the system to move the port locations to the first recommended pose and/or the first recommended port location.

In some embodiments, user selection (1258) of the third interface element causes the robotic system to automatically execute a first movement to move the port location from the actual port location to the first recommended port location.

In some embodiments, generating the recommended pose comprises determining (1260) whether the recommended pose meets a boundary condition.

In some embodiments, the boundary condition is based on a respective location of one or more accessories and/or patient fixation.

In some embodiments, in accordance with a determination (1262) that the recommended pose does not meet the boundary condition (e.g., steps 922 and 924, FIG. 29), the robotic system generates (1264) and displays on the user interface a third notification that includes information of the difference between the actual pose and the first recommended pose exceeds the boundary condition.

In some embodiments, the robotic system also generates (1266) and displays a request to adjust the kinematic chain. In some embodiments, the request comprises a request to the user to manually adjust the kinematic chain (e.g., step 926, FIG. 29). For example, in some embodiments, if a bar (and its associated arms are not within an allowed boundary, the robotic system generates and displays a notification to the user requesting that the user take actions to modify the bar position. In some embodiments, the robotic system notifies the user of actual versus recommended port locations, and/or bar positions, etc., thus enabling the user to be able to make informed decisions on whether to make further adjustments, or proceed with a surgical procedure in a safe manner, etc. The advantage of such features is that the user can be notified of actual v. recommended port locations, bar positions, etc., and can then decide whether to make further adjustments or proceed with a surgical procedure in a safe manner.)

In some embodiments, in accordance with a determination (1268) that the recommended pose exceeds the boundary condition, the robotic system generates and executes a modified target pose that meets the boundary condition.

### 3. Implementing Systems and Terminology.

Embodiments disclosed herein provide systems, methods and apparatus for establishing improved procedural setup.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions for establishing procedural setup described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the embodiments shown herein but is to be accorded the widest scope consistent with the principles and novel features disclosed herein.

Some embodiments or implementations are described with respect to the following clauses:
Clause 1. A robotic system, comprising:
a kinematic chain including at least a first robotic arm;
one or more processors; and memory storing instructions that, when executed by the one or more processors, cause the one or more processors to:
execute first movement of the kinematic chain to a first pose in accordance with a first recommended pose corresponding to a first procedure to be performed on a patient;
after the kinematic chain reaches the first pose by executing the first movement, obtain first data corresponding to a boundary condition of the kinematic chain in accordance with an input from a user and/or second data corresponding to a current state of the patient; and adjust at least a portion of the kinematic chain from the first pose to a second pose in accordance with the obtained first data and/or second data.
Clause 2. The robotic system of clause 1, wherein the memory further stores one or more recommended poses for the kinematic chain, corresponding to one or more procedural setups, the one or more recommended poses including the first pose.
Clause 3. The robotic system of clause 2, wherein the memory further includes instructions that, when executed by the one or more processors, cause the one or more processors to: prior to storing the one or more recommended poses, generate the one or more recommended poses.
Clause 4. The robotic system of any of clauses 1-3, wherein the memory further includes instructions that, when executed by the one or more processors, cause the one or more processors to:
after the kinematic chain reaches the first pose by executing the first movement, place the first robotic arm in a docked state.
Clause 5. The robotic system of clause 4, wherein the memory further includes instructions that, when executed by the one or more processors, cause the one or more processors to: in accordance with a determination that the first robotic arm is in the docked state, determine a port of entry and/or remote center of motion (RCM) with respect to the first robotic arm.
Clause 6. The robotic system of any of clauses 1-5, wherein the boundary condition of the kinematic chain is established by a user.
Clause 7. The robotic system of clause 6, wherein the boundary condition of the kinematic chain is determined based on a respective position of one or more accessories in a vicinity of the kinematic chain.
Clause 8. The robotic system of clause 6 or 7, wherein the boundary condition of the kinematic chain is determined based on a respective position of one or more accessories attached to a support platform of the robotic system.
Clause 9. The robotic system of any of clauses 1-8, wherein the first data corresponding to the boundary condition of the kinematic chain is based on positioning and/or fixation of the patient on the robotic system.
Clause 10. The robotic system of clause 9, wherein the positioning and/or fixation of the patient is inferred from one or more port locations corresponding to the kinematic chain.
Clause 11. A robotic system, comprising:
a display;
a kinematic chain;
one or more processors; and memory storing instructions that, when executed by the one or more processors, cause the one or more processors to:
after the kinematic chain has entered a docked state, determine an actual pose of the kinematic chain and/or an actual port location corresponding to the kinematic chain;
generate a recommended pose for the kinematic chain and/or a first recommended port location corresponding to the kinematic chain in accordance with a first procedural selection; compare the recommended pose with the actual pose and/or the first recommended port location with the actual port location;
in accordance with the comparison, determine that a difference between the recommended pose and the actual pose and/or a difference between the first recommended port location and the actual port location meet first criteria, the first criteria including a first threshold amount of difference; generate a first notification regarding the difference between the recommended pose and the actual pose and/or the difference between the first recommended port location and the actual port location; and
output the first notification.
Clause 12. The robotic system of clause 11, wherein the kinematic chain includes a first robotic arm that is in the docked state.
Clause 13. The robotic system of clause 11 or 12, wherein:
generating the first notification includes generating a first visualization that includes (1) the actual pose and the recommended pose and/or (2) the actual port location and the first recommended port location; and
the memory further includes instructions that, when executed by the one or more processors, cause the one or more processors to:
display the first visualization on a user interface of the robotic system.
Clause 14. The robotic system of any of clauses 11-13, wherein comparing the first recommended port location with the actual port location includes:
determining a separation distance between the first recommended port location and the actual port location; and comparing the separation distance with one or more preset margins.
Clause 15. The robotic system of clause 14, wherein the one or more preset margins include a first preset margin and a second preset margin.
Clause 16. The robotic system of clause 15, wherein the first preset margin is between 3 and 10 millimeters.
Clause 17. The robotic system of clause 15 or 16, wherein the second preset margin is between 0 and 6 millimeters.
Clause 18. The robotic system of any of clauses 15-17, wherein the memory further includes instructions that, when executed by the one or more processors, cause the one or more processors to:
in accordance with a determination that the separation distance exceeds the first preset margin, display on a user interface a first recommendation to manually adjust a port location from the actual location to a first location that is within the first preset margin.
Clause 19. The robotic system of any of clauses 15-18, wherein the memory further includes instructions that, when executed by the one or more processors, cause the one or more processors to:
in accordance with a determination that the separation distance is within the first preset margin, determine whether movement of the kinematic chain from the actual pose to the recommended pose is within a pre-established movement boundary;
in accordance with a determination that the movement of the kinematic chain from the actual pose to the recommended pose exceeds the pre-established movement boundary, display on a user interface a second recommendation for a user to manually adjust the kinematic chain from the actual pose to a second pose; and
in accordance with a determination that the movement of the kinematic chain from the actual pose to the recommended pose is within the pre-established movement boundary, determine whether the separation distance is within the second preset margin.
Clause 20. The robotic system of clause 19, wherein the memory further includes instructions that, when executed by the one or more processors, cause the one or more processors to:
in accordance with a determination that the separation distance is within the second preset margin: generate a second visualization that includes the actual port location and the first recommended port location;
generate a second notification requesting user confirmation that it is safe to move a port location from the actual port location to the first recommended port location; and
display the second visualization, the second notification, and a first interface element corresponding to the second notification on the user interface.
Clause 21. The robotic system of clause 20, wherein the memory further includes instructions that, when executed by the one or more processors, cause the one or more processors to:
receive user selection of the first interface element; and
in response to the user selection, generate and display, on the user interface, a second interface element,
wherein user selection of the second interface element causes the robotic system to automatically execute a first movement to move the port location from the actual port location to the first recommended port location.
Clause 22. The robotic system of clause 21, wherein:
the kinematic chain comprises a first robotic arm that is in the docked state; and
the memory further includes instructions that, when executed by the one or more processors, cause the one or more processors to:
in accordance with executing the first movement to move the port location from the actual port location to the first recommended port location, adjust a remote center of motion (RCM) with respect to the first robotic arm based on the first recommended port location.
Clause 23. The robotic system of clause 22, wherein adjusting the remote center of motion (RCM) comprises moving at least a portion of the first robotic arm and/or an adjustable bar of the kinematic chain relative to a support platform of the robotic system.
Clause 24. The robotic system of any of clauses 19-23, wherein the memory further includes instructions that, when executed by the one or more processors, cause the one or more processors to:
in accordance with a determination that the separation distance exceeds the second margin, generate and display on the user interface:
a third visualization that includes (1) the actual pose and the first recommended pose and/or (2) the actual port location and the first recommended port location; and a third interface element,
wherein user selection of the third interface element causes the robotic system to automatically execute a first movement to move the port location from the actual port location to the first recommended port location.
Clause 25. The robotic system of any of clauses 11-24, wherein generating the recommended pose comprises determining whether the recommended pose meets a boundary condition.
Clause 26. The robotic system of clause 25, wherein the boundary condition is based on a respective location of one or more accessories and/or patient fixation.
Clause 27. The robotic system of clause 25, wherein the memory further includes instructions that, when executed by the one or more processors, cause the one or more processors to:
in accordance with a determination that the recommended pose does not meet the boundary condition, generate and display on the user interface:
a third notification that includes information of the difference between the actual pose and the first recommended pose exceeds the boundary condition; and a request to adjust the kinematic chain.
Clause 28. The robotic system of clause 25, wherein the memory further includes instructions that, when executed by the one or more processors, cause the one or more processors to: in accordance with a determination that the recommended pose exceeds the boundary condition, generate and execute a modified target pose that meets the boundary condition.

What is claimed is:

1. A robotic system, comprising:
a kinematic chain including at least a first robotic arm;
one or more processors; and
memory storing instructions that, when executed by the one or more processors, cause the one or more processors to:
execute first movement of the kinematic chain to a first pose in accordance with a first recommended pose corresponding to a first procedure to be performed on a patient;
after the kinematic chain reaches the first pose by executing the first movement, obtain first data corresponding to a boundary condition of the kinematic chain in accordance with an input from a user and/or second data corresponding to a current state of the patient; and
adjust at least a portion of the kinematic chain from the first pose to a second pose in accordance with the obtained first data and/or second data.

2. The robotic system of claim 1, wherein the memory further stores one or more recommended poses for the kinematic chain, corresponding to one or more procedural setups, the one or more recommended poses including the first pose.

3. The robotic system of claim 2, wherein the memory further includes instructions that, when executed by the one or more processors, cause the one or more processors to:
prior to storing the one or more recommended poses, generate the one or more recommended poses.

4. The robotic system of claim 1, wherein the memory further includes instructions that, when executed by the one or more processors, cause the one or more processors to:
after the kinematic chain reaches the first pose by executing the first movement, place the first robotic arm in a docked state.

5. The robotic system of claim 4, wherein the memory further includes instructions that, when executed by the one or more processors, cause the one or more processors to:
in accordance with a determination that the first robotic arm is in the docked state, determine a port of entry and/or remote center of motion (RCM) with respect to the first robotic arm.

6. The robotic system of claim 1, wherein the boundary condition of the kinematic chain is established by a user.

7. The robotic system of claim 6, wherein the boundary condition of the kinematic chain is determined based on a respective position of one or more accessories in a vicinity of the kinematic chain.

8. The robotic system of claim 6, wherein the boundary condition of the kinematic chain is determined based on a respective position of one or more accessories attached to a support platform of the robotic system.

9. The robotic system of claim 1, wherein the first data corresponding to the boundary condition of the kinematic chain is based on positioning and/or fixation of the patient on the robotic system.

10. The robotic system of claim 9, wherein the positioning and/or fixation of the patient is inferred from one or more port locations corresponding to the kinematic chain.

11. A robotic system, comprising:
a display;
a kinematic chain;
one or more processors; and
memory storing instructions that, when executed by the one or more processors, cause the one or more processors to:
after the kinematic chain has entered a docked state, determine an actual pose of the kinematic chain and/or an actual port location corresponding to the kinematic chain;
generate a recommended pose for the kinematic chain and/or a first recommended port location corresponding to the kinematic chain in accordance with a first procedural selection;
compare the recommended pose with the actual pose and/or the first recommended port location with the actual port location;
in accordance with the comparison, determine that a difference between the recommended pose and the actual pose and/or a difference between the first recommended port location and the actual port location meet first criteria, the first criteria including a first threshold amount of difference;
generate a first notification regarding the difference between the recommended pose and the actual pose and/or the difference between the first recommended port location and the actual port location; and
output the first notification.

12. The robotic system of claim 11, wherein the kinematic chain includes a first robotic arm that is in the docked state.

13. The robotic system of claim 11, wherein:
generating the first notification includes generating a first visualization that includes (1) the actual pose and the recommended pose and/or (2) the actual port location and the first recommended port location; and
the memory further includes instructions that, when executed by the one or more processors, cause the one or more processors to:
display the first visualization on a user interface of the robotic system.

14. The robotic system of claim 11, wherein comparing the first recommended port location with the actual port location includes:
determining a separation distance between the first recommended port location and the actual port location; and
comparing the separation distance with one or more preset margins.

15. The robotic system of claim 14, wherein the one or more preset margins include a first preset margin and a second preset margin.

16. The robotic system of claim 15, wherein the first preset margin is between 3 and 10 millimeters.

17. The robotic system of claim 15, wherein the second preset margin is between 0 and 6 millimeters.

18. The robotic system of claim 15, wherein the memory further includes instructions that, when executed by the one or more processors, cause the one or more processors to:
in accordance with a determination that the separation distance exceeds the first preset margin, display on a user interface a first recommendation to manually adjust a port location from the actual location to a first location that is within the first preset margin.

19. The robotic system of claim 15, wherein the memory further includes instructions that, when executed by the one or more processors, cause the one or more processors to:
in accordance with a determination that the separation distance is within the first preset margin, determine whether movement of the kinematic chain from the actual pose to the recommended pose is within a pre-established movement boundary;
in accordance with a determination that the movement of the kinematic chain from the actual pose to the recommended pose exceeds the pre-established movement boundary, display on a user interface a second recommendation for a user to manually adjust the kinematic chain from the actual pose to a second pose; and
in accordance with a determination that the movement of the kinematic chain from the actual pose to the recommended pose is within the pre-established movement boundary, determine whether the separation distance is within the second preset margin.

20. The robotic system of claim 19, wherein the memory further includes instructions that, when executed by the one or more processors, cause the one or more processors to:
in accordance with a determination that the separation distance is within the second preset margin:
generate a second visualization that includes the actual port location and the first recommended port location;
generate a second notification requesting user confirmation that it is safe to move a port location from the actual port location to the first recommended port location; and
display the second visualization, the second notification, and a first interface element corresponding to the second notification on the user interface.

21. The robotic system of claim 20, wherein the memory further includes instructions that, when executed by the one or more processors, cause the one or more processors to:
receive user selection of the first interface element; and
in response to the user selection, generate and display, on the user interface, a second interface element,
wherein user selection of the second interface element causes the robotic system to automatically execute a first movement to move the port location from the actual port location to the first recommended port location.

22. The robotic system of claim 21, wherein:
the kinematic chain comprises a first robotic arm that is in the docked state; and
the memory further includes instructions that, when executed by the one or more processors, cause the one or more processors to:
in accordance with executing the first movement to move the port location from the actual port location to the first recommended port location, adjust a remote center of motion (RCM) with respect to the first robotic arm based on the first recommended port location.

23. The robotic system of claim 22, wherein adjusting the remote center of motion (RCM) comprises moving at least a portion of the first robotic arm and/or an adjustable bar of the kinematic chain relative to a support platform of the robotic system.

24. The robotic system of claim 19, wherein the memory further includes instructions that, when executed by the one or more processors, cause the one or more processors to:
in accordance with a determination that the separation distance exceeds the second margin, generate and display on the user interface:
a third visualization that includes (1) the actual pose and the first recommended pose and/or (2) the actual port location and the first recommended port location; and
a third interface element,
wherein user selection of the third interface element causes the robotic system to automatically execute a first movement to move the port location from the actual port location to the first recommended port location.

25. The robotic system of claim 11, wherein generating the recommended pose comprises determining whether the recommended pose meets a boundary condition.

26. The robotic system of claim 25, wherein the boundary condition is based on a respective location of one or more accessories and/or patient fixation.

27. The robotic system of claim 25, wherein the memory further includes instructions that, when executed by the one or more processors, cause the one or more processors to:
in accordance with a determination that the recommended pose does not meet the boundary condition, generate and display on the user interface:
a third notification that includes information of the difference between the actual pose and the first recommended pose exceeds the boundary condition; and
a request to adjust the kinematic chain.

28. The robotic system of claim 25, wherein the memory further includes instructions that, when executed by the one or more processors, cause the one or more processors to:
in accordance with a determination that the recommended pose exceeds the boundary condition, generate and execute a modified target pose that meets the boundary condition.
